(19) United States
(12) Patent Application Publication
(10) Pub. No.: US 2014/0328838 A1
(43) Pub. Date: Nov. 6, 2014

(54) CANINISED ANTIBODIES AND METHOD FOR PRODUCTION OF SAME

(71) Applicant: David Gearing, Melbourne (AU)

(72) Inventor: David Gearing, Melbourne (AU)

(73) Assignee: NVIP Pty Ltd

(21) Appl. No.: 14/342,943

(22) PCT Filed: Sep. 5, 2012
(86) PCT No.: PCT/GB2012/052174

§ 371 Date: Jun. 16, 2014

Related U.S. Application Data
(60) Provisional application No. 61/531,439, filed on Sep. 6, 2011.

(51) Int. Cl. C07K 16/46; G01N 33/68; G06F 19/18

(52) U.S. Cl. CPC C07K 16/461; G06F 19/18; G01N 33/6854
USPC 424/133.1; 506/8; 530/387.3

(57) ABSTRACT

A method of producing a non-immunogenic immunoglobulin for administration to a target species is provided wherein the method comprises substituting amino acid residues in framework regions of a donor immunoglobulin with amino acid residues present at a corresponding position in framework regions of at least one immunoglobulin derived from the target species. Also provided are antibodies produced by the method of the invention, including novel humanised and caninised anti-NGF antibodies. The invention extends to nucleic acids encoding same and to methods of treating pain and arthritis in a human or dog using said antibodies and/or nucleic acids.

### FIELD OF THE INVENTION

The present invention relates to methods for producing non-immunogenic binding agents, in particular immunoglobulins and fragments thereof, which can be used in therapeutic methods. In particular, the methods allow a donor immunoglobulin to be modified such that it can be administered to a target species with minimal risk of neutralising antibodies being raised there against. The invention further extends to immunoglobulins produced by said methods and to their use in therapy.

### BACKGROUND TO THE INVENTION

Advances in recombinant DNA technology have resulted in a variety of proteins being developed for pharmaceutical applications. This has resulted in a number of protein-based drugs, which may be more generally referred to as biologics, being the subject of clinical trials or market approval. Due to their inherent properties and structures, protein-based drugs are significantly larger and more complex than more traditional small molecule organic and inorganic based molecules. In particular, the folded tertiary structure of the protein is essential to its biological function.

Antibodies are one family of proteins that are being widely developed and used in therapeutic applications in man. The popularity of antibodies in therapeutic applications has been due to their versatility in being able to specifically target virtually any desired target molecule. The majority of protein based therapeutic products are monoclonal antibodies. However one significant drawback associated with the use of monoclonal antibody-based therapies is the production of neutralising antibodies against the therapeutic monoclonal antibody when administered to a subject. These neutralising antibodies result from the immune system of the subject recognising as foreign sequences of amino acids that are present in the administered monoclonal antibody. As a result, an immune response is mounted against the administered therapeutic antibody. The production of neutralising antibodies by the subject can significantly impair the ability to continue treating the subject with the therapeutic monoclonal antibody. Typically, once neutralising antibodies have been produced in the subject, the use of the therapeutic antibody needs to be increased as the neutralising antibodies effectively reduce or negate the therapeutic effect of the administered monoclonal antibody. This can limit the use of the therapeutic antibody to an initial treatment only, with the result that repeat or long term dosing of the therapeutic antibody is not an option. In short, the production of neutralising antibodies against a therapeutic antibody can significantly limit the therapeutic use of that antibody and this, in turn can significantly limit, or completely prevent, the use of the antibody in treatment of chronic or recurring diseases.

To attempt to reduce the likelihood of neutralising antibodies being produced against a therapeutic antibody, a number of approaches have been developed which are designed to reduce the immunogenicity of the antibody by modifying its structure. One such approach is the production of a chimeric antibody whereby the heavy and light chain constant domains of the antibody are derived from an antibody obtained from the same species as the subject to whom the antibody is to be administered. As most therapeutic antibodies have been developed for use in humans, such chimeric antibodies typically comprise human derived heavy and light chain constant domains conjoined to heavy and light chain variable regions derived from a non-human antibody, most typically of mouse or rat origin.

Further approaches to reduce the immunogenicity of therapeutic antibodies employ techniques referred to as humanisation. The term humanisation reflects the fact that the modified antibodies are being made more like antibodies which would be produced by a human, in order to limit the possibility of an immune response resulting. Humanisation techniques extend to a number of different approaches to making an antibody more human-like.

One commonly used humanisation technique is that of CDR grafting whereby the complementarity determining regions (CDRs) derived from a donor antibody, such as a murine derived antibody, are combined with framework regions derived from an antibody of human origin to form heavy and light chain variable domains which are then combined with human antibody derived heavy and light chain constant domains. The resulting antibody therefore contains only a limited number of non-human derived amino acids, this serving to limit the presence of epitopes which will be viewed by the human immune system as foreign.

One significant drawback associated with humanisation is that it often results in a significant reduction in the binding affinity of the resulting humanised antibody over that exhibited by the non-humanised donor antibody. Just as is the case with the production of neutralising antibodies against a therapeutic monoclonal antibody, this can result in the therapeutic use of that antibody being significantly compromised. In particular, the reduction in binding affinity results in the need to administer a greater dose of the therapeutic antibody and it may further require the dosage frequency to be increased too. Both of these factors result in an increase in the cost of therapy and an increase in the inconvenience to the patient. Further, the administration of larger quantities of the antibody to a subject results in an increased risk that neutralising antibodies will be raised against the therapeutic antibody.

In cases where the humanised antibodies bind to the desired epitope with lower affinity than the initial donor antibody, this can be due to the incompatible structural alignment of framework region sequences with the epitope binding CDR sequences. Through an iterative process of back-mutating human residues with the amino acids at the same position in the donor antibody, the affinity of the humanised antibody can often be restored. Although the back mutation process can result in further non-human amino acid residues being reintroduced into the humanised antibody, the resulting antibody is still termed humanised despite the presence of significant donor amino acids in the final sequence.

By direct analogy, the speciesisation of antibodies for use in species other than humans is similarly compromised by the requirement to make framework changes that maintain the binding specificity of the modified antibody, while reducing the immunogenicity of the resultant antibody in the target species of choice. From a commercial perspective, the use of antibodies in species other than humans is desirable because of the range of common disease processes that could usefully be treated, in particular, in high-value species such as companion animals (cats and dogs) and endurance animals (horses and camels), or to improve meat quality in food producing animals, such as cows, sheep, pigs and chickens. Accordingly, methods that enable simpler conversion of antibodies for use in these species would be highly desirable.

There is therefore a need for improved methods of modifying a donor antibody in order that it can be administered to a target subject without the resulting antibody exhibiting a loss in binding affinity to the target antigen, while minimizing changes from those of the recipient antibody sequences in order to reduce the likelihood of neutralising antibodies being raised there against. It follows that a method that converts a donor antibody sequence to a target species sequence with a minimum number of changes to achieve a target species framework structure with minimum impact on CDR structure would be highly desirable.

### SUMMARY OF THE INVENTION

The present invention describes methods to modify a donor antibody for use in a target species so that the resultant antibody does not contain any amino acid at any position within the framework regions which would be foreign at that position in that species. The modified antibody will therefore retain the specificity and affinity of the donor antibody, but at the same time will be modified so that no potentially foreign epitopes will be created. The modified antibody will therefore not be seen as foreign in the target species and hence will not induce an immune response which could lead to a neutralisation of its efficacy, especially following long term administration. The method whereby this can be achieved overcomes all the disadvantages inherent in earlier methods and yet is characterised by a remarkable simplicity and elegance.

Following extensive efforts, the present inventor has surprisingly developed a method for altering a donor antibody or an antigen binding fragment derived therefrom such that it is completely or significantly non-immunogenic when administered to a target species, said target species being a different species to that from which the donor antibody was derived. Typically, neutralising antibodies are not raised against the resulting antibody following administration to a subject.

Furthermore, the alteration of the antibody to make it non-immunogenic would not result in a reduction in binding specificity or affinity to the intended target.

According to a first aspect of the present invention there is provided method of producing a non-immunogenic immunoglobulin for administration to a target species, the method comprising the steps of:
identifying a donor immunoglobulin from a species other than the target species, wherein the donor immunoglobulin has binding specificity to a target epitope present in the target species,
determining an amino acid sequence of framework regions of heavy and/or light chain variable domains of the donor immunoglobulin,
comparing each amino acid residue of the amino acid sequence of the framework regions of the heavy and/or light chain variable domains of the donor immunoglobulin with an amino acid residue present at a corresponding position in an amino acid sequence of framework regions of one or more immunoglobulins derived from the target species to identify one or more amino acid residues within the amino acid sequence of the framework regions of the heavy and/or light chain variable domains of the donor immunoglobulin that is not present at the corresponding position in the amino acid sequence of the framework regions of at least one of the one or more immunoglobulins derived from the target species, and
substituting the one or more identified amino acid residues present in the amino acid sequence of the framework regions of the heavy and/or light chain variable domains of the donor immunoglobulin, but not present at the corresponding position in the amino acid sequence of the framework regions of at least one of the one or more immunoglobulins derived from the target species, with an amino acid residue which is present at the corresponding position in the amino acid sequence of the framework regions of at least one of the one or more immunoglobulins derived from the target species.

The method leaves unaltered (i.e. unsubstituted) any amino acid which is present at a specific framework (FW) region position in the donor immunoglobulin which is also present in at least one of the corresponding framework region positions in immunoglobulins derived from the target species. In certain embodiments, the amino acid sequences of the framework regions of the immunoglobulins of the target species comprise a pool of residues which can be compared to the amino acid residue present at the corresponding position in the donor immunoglobulin. Typically such a pool comprises positional specific amino acid residues derived from a plurality of target species immunoglobulins. Typically the pool comprises framework region sequence data derived from as many immunoglobulins of a target species as possible, and, if viable, all framework region sequences of all immunoglobulins known for a particular species which are present in published databases.

The amino acid sequences of the framework regions of immunoglobulins derived from different species can be derived from a number of publically available databases which will be well known to the person skilled in the art. For example, the databases held by the National Center for Biotechnology Information (NCBI) contain immunoglobulin sequence information for antibodies derived from a wide variety of species. Further databases may include any database which comprises publicly available germline and expressed cDNA sequences and may include journal publications or databases such as, but not limited to, the Kabat database of immunoglobulin sequences (URL: www.kabatdatabase.com) and V BASE, the human antibody database (http://vbase.mrc-cpe.cam.ac.uk/). Procedures to prepare a table of possible target amino acids is routine to the skilled person.

Although the comparison may be between the donor sequence and a single member of the target sequence, it will be obvious that comparison with a pool of target sequences is preferred because this will expand the number of natural options at each Kabat position in the target species. Not only will this increase the chance of a “match” between the donor and the target, but it will also expand the options for replacement where a match does not exist.

As herein defined, a non-immunogenic immunoglobulin is an immunoglobulin which does not have an immune response raised there against when it is administered to a target species. In particular, a humoral (antibody mediated) response is not mediated against the antibody, particularly against epitopes comprising amino acid residues derived from the framework (FW) regions.

Where donor and target amino acids differ at any Kabat number, the amino acid must be chosen from one known to be natural at that position in the target. This will lead to a number of possible sequences, any of which may lead to a preferred or at least suitable sequence of the invention. Where substitution of an amino acid residue present in a donor immunoglobulin framework region is required, typically this is undertaken using the principle of conservative substitution. Typically, conservative substitution requires replacement of the amino acid with a homologous amino acid residue, that is a residue which shares similar characteristics or properties. Such a replacement may be known as homologous substitution.

In determining whether a substituted amino acid can be replaced with a conserved amino acid, an assessment may typically be made of factors such as, but not limited to, (a) the structure of the polypeptide backbone in the area of the substitution, for example, a sheet or helical conformation, (b) the charge or hydrophobicity of the molecule at the target site, and/or (c) the bulk of the side chain(s). If a residue can be substituted with a residue which has common characteristics, such as a similar side chain or similar charge or hydrophobicity, then such a residue is preferred as a substitute.

When considering whether a donor immunoglobulin derived residue which is not present in the pool of target species immunoglobulin residues present at a corresponding position can be conservatively substituted, it may be preferable to assess whether a homologous amino acid is available in the pool of corresponding residues derived from the target species for that specific position based on the amino acids being grouped together according to similarities in the properties of their side chains (A. L. Lehninger, in Biochemistry, 2^{nd} Ed., 73-75, Worth Publishers, New York (1975)). For example, the following groups can be determined: (1) non-polar: Ala (A), VaI (V), Leu (L), Ile (I), Pro (P), Phe (F), Trp (W), Met (M); (2) uncharged polar: GIy (G), Ser (S), Thr (T), Cys (C), Tyr (Y), Asn (N), Gln (O); (3) acidic: Asp (D), GIu (E); and (4) basic: Lys (K), Arg (R), His (H). Hence the substitution of an amino acid residue with another present in the same group is preferred.

Alternatively, the amino acids may be grouped as follows: (1) aromatic: Phe (F), Trp (W), Tyr (Y); (2) apolar: Leu (L), Val (V), Ile (I), Ala (A), Met (M); (3) aliphatic: Ala (A), Val (V), Leu (L), Ile (I); (4) acidic: Asp (D), Glu (E); (5) basic: His (H), Lys (K), Arg (R); and (6) polar: Gln (O), Asn (N), Ser (S), Thr (T), Tyr (Y). Again, the substitution of an amino acid residue with another present in the same group is preferred.

Alternatively, amino acid residues may be divided into groups based on common side-chain properties: (1) hydrophobic: Met (M), Ala (A), VaI (V), Leu (L), Ile (1); (2) neutral hydrophilic: Cys (C), Ser (S), Thr (T), Asn (N), Gin (O); (3) acidic: Asp (D), GIu (E); (4) basic: His (H), Lys (K), Arg R); (5) residues that influence chain orientation: GIy (G), Pro (P); and (6) aromatic: Trp (W), Tyr (Y), Phe (F). Again, the substitution of an amino acid residue with another present in the same group would be preferred.

Accordingly, conservative substitutions will entail exchanging (substituting) a member of one of these classes for another member of that same class. In certain embodiments, the amino acid residue which is introduced into the donor immunoglobulin framework region sequence is the consensus amino acid defined at that specific position from the pool of immunoglobulins derived from the target species. The consensus amino acid is the amino acid which is most commonly found at that position in immunoglobulins which comprise the collection of target species immunoglobulins which contribute to the pool.

Typically the substitution of the framework region residues does not result in a reduction in the binding of the immunoglobulin to its intended ligand. In particular, there is no reduction in the binding affinity or specificity.

In certain embodiments the method further comprises the step of replacing at least one and preferably all of the constant domains of the heavy and/or light chain of the donor immunoglobulin with equivalent heavy and/or light chains derived from an immunoglobulin derived from the target species. In certain embodiments, the target species derived constant domains are of the antibody subtype Immunoglobulin G (IgG).

In certain embodiments, the pool of donor immunoglobulin amino acid sequences used to assess the amino acid residues of the framework regions may be restricted to sub-types of immunoglobulins derived from the target species, e.g. from kappa or lambda light chains.

Without wishing to be bound by theory, the method results in only a minimum number of essential changes (amino acid substitutions) being made to the donor immunoglobulin framework region sequences, whilst ensuring that all of the amino acids in the resulting framework region sequence align with those of the target species. This consequently minimises structural changes resulting from the modification of the antibody from a donor immunoglobulin to an antibody which will be completely or substantially non-immunogenic when administered to a target subject. Due to the method involving the substitution of the fewest possible residues, the method may be referred to as Parsimonious Essential Translation (PET). By extension, the changes made to a donor immunoglobulin to allow it to be non-immunogenic when administered to a target animal species, may be referred to as PETisation and the resulting antibody may be referred to as being PETized. This process may, for example, result in the speciesisation of an existing human, mouse or rat antibody such that it may be administered to another target species, in particular an animal species, such as human, dog, cat, or horse without neutralising antibodies being raised there against.

In certain embodiments, the target species is a mammalian target species. In one embodiment, the target mammalian species is an animal, in particular a companion animal such as, but not limited to, a dog, cat or horse or a livestock animal. In further embodiments, the mammalian target species is a human.

In certain embodiments, the target epitope is nerve growth factor (NGF) or tumour necrosis factor (TNF).

In various further embodiments there is provided a composition containing an immunoglobulin provided by the method of a foregoing aspect of the invention, or an antigen binding fragment thereof. The composition may further comprises at least one pharmaceutically acceptable diluent or carrier. A yet further aspect provides use of an immunoglobulin produced by the method of a foregoing aspect of the invention, or an antigen binding fragment thereof, in the preparation of a medicament for the treatment or prevention of disease. In various further aspects, the present invention extends to the use of the foregoing immunoglobulins, or an antigen binding fragment thereof, in therapeutic and diagnostic methods. The invention further extends to an immunoglobulin produced according to any one of claims 1 to 15, or an antigen binding fragment thereof, for use in the treatment or prevention of disease.

A yet further aspect of the invention relates to the administration of an immunoglobulin produced in accordance with any of the methods defined herein, or an antigen binding fragment thereof, to a subject, in particular a mammalian subject, for the treatment or prevention of disease.

### De-Immunisation

In various further aspects, the invention extends to the modification of a therapeutic immunoglobulin in order to render it non-immunogenic when administered to a specific species. Said modification may be applied to a chimeric antibody, an antibody produced by a CDR grafting technique or a humanised antibody.

In a further aspect, the invention therefore provides a method for modifying a therapeutic immunoglobulin to make it non-immunogenic, the method comprising the steps of:
providing a donor therapeutic immunoglobulin,
determining an amino acid sequence of at least one framework region of variable domains of light and/or heavy chains of the donor immunoglobulin
obtaining a pool of amino acid sequences relating to at least one framework region of variable domains of light and/or heavy chains of immunoglobulins derived from a target species to which the immunoglobulin is to be administered,
comparing amino acid residues of the amino acid sequence of the at least one framework region from the light and/or heavy chains of the donor immunoglobulin to amino acid residues having the same Kabat numbering in the pool of amino acid sequences, and
substituting any amino acid residue of the amino acid sequence of the at least one framework region from the light and/or heavy chains of the donor immunoglobulin with an amino acid residue having the same Kabat numbering in the pool of amino acid sequences where the amino acid residue present in the amino acid sequence of the at least one framework region from the light and/or heavy chains of the donor immunoglobulin differs from the amino acid residues having the same Kabat numbering in the pool of amino acid sequences.

Typically re-humanising therapeutic immunoglobulins using this methodology results in an immunoglobulin that is less immunogenic than the unaltered donor therapeutic antibody. Furthermore, the modified antibody retains its binding affinity and specificity. Hence, the resulting modified antibody is more therapeutically useful.

In certain embodiments, the resulting PETised antibody, or binding fragment thereof, binds to the desired target epitope with a binding affinity K_{D} of 1×10⁻⁸ or less.

A yet further aspect of the invention extends to the provision of at least one framework region for use in an immunoglobulin heavy and/or light chain variable domain. The method of this aspect of the invention may have particular utility in a process such as humanisation of an antibody, or in an equivalent process used to modify an antibody to de-immunise it prior to its administration to a species other than a human. The framework regions provided by this aspect of the invention may be introduced into an antibody which is either about to undergo humanisation or a similar speciesisation process, or can be retrospectively introduced into an antibody which has previously undergone speciesisation. Specifically, the modified framework regions may be introduced into an antibody which has undergone, or which is about to undergo, modification by virtue of a process such as CDR grafting, or which is a chimeric antibody wherein the Fab region of the antibody is derived from a first species and the Fc region of the antibody is derived from a second species.

Accordingly, this further aspect provides a method of modifying an amino acid sequence of at least one framework region of a donor immunoglobulin heavy and/or light chain variable domain, said method comprising the steps of:
determining the amino acid sequence of the at least one framework region sequence of the donor immunoglobulin;
on a residue by residue basis, comparing specific amino acid residues at each position of the at least one framework region of the donor immunoglobulin to a database comprising a pool of amino acid residues found at a corresponding amino acid position in framework region sequences found in antibodies derived from a species to whom the modified source immunoglobulin is to be administered;
substituting any amino acid residues which are present at a specific position in the at least one framework region of the donor immunoglobulin, but not in the pool of amino acid residues found at the corresponding amino acid position, with an amino acid residue which is present in the pool of amino acid residues found at the corresponding amino acid position; and
leaving unaltered any amino acid residues which are present at a specific position in the at least one framework region of the donor immunoglobulin and also present in the pool of amino acid residues found at the corresponding amino acid position.

Typically, any replaced amino acid residues are substituted with an amino acid residue which is the most homologous to the amino acid residue being replaced. As defined hereinbefore, homologous groups of amino acid residues are known. If a homologous amino acid residue is not present in the pool, then the amino acid may be substituted with the amino acid residue which occurs most frequently at that specific position, the so called consensus amino acid residue.

In certain embodiments, the method extends to a method for producing a modified antibody which comprises the steps of expressing the modified framework region sequence along with complementarity determining regions (CDRs) and heavy and/or light chain constant domains such that a heterotetrameric antibody is produced comprising said modified framework region sequences.

Certain further aspects of the present invention extend to providing an oligonucleotide which expresses the amino acid sequence of the modified framework region sequence and to the expression of same in a host cell.

A yet further aspect of the present invention relates to a method for producing a therapeutic antibody with non-immunogenic framework region sequences, said method comprising the steps of:
identifying framework region amino acid residues which are to be substituted by comparing amino acid sequences of framework regions to pools of corresponding amino acid residues present at corresponding amino acid positions in a plurality of immunoglobulins derived from a species to which the therapeutic antibody is to be administered to identify one or more amino acid residues which differ at a specific position; and
substituting the one or more identified amino acid residues with an amino acid residue present in the pool of corresponding amino acid residues.

In certain embodiments the identification of whether a framework region amino acid residue is present in the corresponding positional pool of amino acid residues from the species to which the antibody will be administered is achieved by performing an alignment of the sequence and the pool residues. Essentially, the residues which are substituted do not reduce the binding activity of the resulting modified antibody. That is, the amino acid which is substituted may be substituted for a different amino acid without significantly affecting the binding characteristics of the antibody. This is primarily achieved by substituting the amino acid with a homologous amino acid, that is, an amino acid with similar or related characteristics, such as size, polarity/charge or hydrophobicity of the molecule at the target site, or the bulk of the side chain. Alternatively, the residue may be substituted with the consensus residue which occurs in the target species at a corresponding position.

The amino acid residues which are substituted (or substitutable) are present at positions which may be known as variant tolerant positions. That is, the substitution of that residue for another residue does not alter the binding specificity of the complementarity determining regions which are interposed between the framework regions. Such a substitution may be deemed necessary due to the fact that a residue present at a specific position of a framework region in one species may be absent at a corresponding position in the framework region sequences of a second species. Hence, the amino acid may cause an immunogenic response to be raised there against by virtue of forming an epitope which is viewed as foreign by the immune system of a species where that amino acid residue is not normally present at that position of a framework region sequence. Using the methodology of substituting that outlier residue with a homologous residue or consensus residue which is present in the target species, the potentially foreign epitope can be altered to form an epitope which will not be recognised as foreign. Removal of all such epitopes from the framework region of an antibody can therefore prevent a humoral response being raised against that portion of the antibody when administered to a subject which is of a different species to the species from which the antibody was initially derived.

### Ubiquitous Species-Specific Framework Region Production

The inventor has further defined a series of framework regions (FR) which can be combined with complementarity determining regions (CDRs) to form non-immunogenic PETised heavy and light chain variable domains. Each of the heavy and light chain domains has 4 framework regions, designated FR1, FR2, FR3 and FR4. This methodology can be applied to de-immunise any antibody (immunoglobulin) in order that it can be administered to a desired species. However, for the purposes of exemplification only, the undernoted examples will illustrate the production and use of such framework regions in human, canine, feline and equine-based antibodies.

### Antibody Structure

An antibody molecule may comprise a heavy chain variable domain comprising CDR1, CDR2 and CDR3 regions and associated interposed framework regions. The heavy chain variable domain (VH) CDRs are known as VHCDRs, with these CDRs being found at the following positions according to the Kabat numbering system: VHCDR1—Kabat residues 31-35, VHCDR2—Kabat residues 50-65 and VHCDR3—Kabat residues 95-102 (Kabat E A et al. (1991) Sequences of proteins of immunological interest, 5^{th} edition. Bethesda: US Department of Health and Human Services).

Furthermore, an antibody further comprises a light chain variable domain comprising CDR1, CDR2 and CDR3 regions and associated interposed framework regions. The light chain variable domain (VL) CDRs are known as VLCDRs, with these CDRs being found at the following amino acid residue positions according to the Kabat numbering system: VLCDR1—Kabat residues 24-34, VLCDR2—Kabat residues 50-56 and VLCDR3—Kabat residues 89-97.

A light or heavy chain variable domain comprises four framework regions, FR1, FR2, FR3 and FR4, interposed with CDRs in the following arrangement: FR1-CDR1-FR2-CDR2-FR3-CDR3-FR4.

A yet further aspect of the present invention provides an immunoglobulin for administration to a target species, comprising:
light and heavy chain constant domains derived from an immunoglobulin of the target species,
complementary determining regions (CDRs) derived from a donor immunoglobulin wherein the donor immunoglobulin specifically binds to a ligand which is present in the target species, and
framework regions derived from the donor immunoglobulin wherein any amino acid residues not also present at a corresponding position in any immunoglobulin from the target species are substituted with an amino acid which is found in the corresponding position in the target species.

Typically no amino acid substitutions are made to the CDR regions of the antibody. Furthermore, in certain embodiments the framework regions comprise no more than 7 consecutive amino acid residues being substituted with residues from the target species. Furthermore, in certain embodiments the framework regions comprise no more than 5 consecutive amino acid residues being substituted with residues from the target species. Furthermore, in certain embodiments the framework regions comprise no more than 3 consecutive amino acid residues being substituted with residues from the target species.

Furthermore, in certain embodiments no more than 10 amino acid residues of the donor immunoglobulin framework region (heavy chain FR1, FR2, FR3 and FR4 and light chain FR1, FR2, FR3 and FR4) are substituted with residues from the target species. Furthermore, in certain embodiments no more than 7 amino acid residues of the donor immunoglobulin framework region (heavy chain FR1, FR2, FR3 and FR4 and light chain FR1, FR2, FR3 and FR4) are substituted with residues from the target species. Furthermore, in certain embodiments no more than 5 amino acid residues of the donor immunoglobulin framework region (heavy chain FR1, FR2, FR3 and FR4 and light chain FR1, FR2, FR3 and FR4) are substituted with residues from the target species.

In certain embodiments the target species is a mammalian species. In particular the target species may be a human, canine, feline or equine.

Accordingly, in embodiments where the immunoglobulin is to be administered to a canine as the target species, the immunoglobulin comprises constant domain regions derived from a canine derived antibody or antibodies, and the residues substituted into the donor immunoglobulin framework regions are derived from corresponding canine framework regions amino acid residues.

In embodiments where the immunoglobulin is to be administered to a feline as the target species, the immunoglobulin comprises constant domain regions derived from a feline derived antibody or antibodies, and the residues substituted into the donor immunoglobulin framework regions are derived from corresponding feline framework regions amino acid residues.

In embodiments where the immunoglobulin is to be administered to an equine as the target species, the immunoglobulin comprises constant domain regions derived from an equine derived antibody or antibodies, and the residues substituted into the donor immunoglobulin framework regions are derived from corresponding equine framework regions amino acid residues.

In embodiments where the immunoglobulin is to be administered to a human as the target species, the immunoglobulin comprises constant domain regions derived from a human derived antibody or antibodies, and the residues substituted into the donor immunoglobulin framework regions are derived from corresponding human framework regions amino acid residues.

In various further embodiments there is provided a composition containing an immunoglobulin of the foregoing aspect of the invention, or an antigen binding fragment thereof. The composition may further comprises at least one pharmaceutically acceptable diluent or carrier. A yet further aspect provides use of an immunoglobulin of the foregoing aspect of the invention, or an antigen binding fragment thereof, in the preparation of a medicament for the treatment or prevention of disease. In various further aspects, the present invention extends to the use of the foregoing immunoglobulins, or an antigen binding fragment thereof, in therapeutic and diagnostic methods. The invention further extends to an immunoglobulin of the foregoing aspect of the invention, or an antigen binding fragment thereof, for use in the treatment or prevention of disease.

A yet further aspect of the invention relates to the administration of an immunoglobulin of the foregoing aspect of the invention, or an antigen binding fragment thereof, to a subject, in particular a mammalian subject, for the treatment or prevention of disease.

A yet further aspect of the present invention provides a method of producing an immunoglobulin comprising the steps of:
introducing nucleotides encoding heavy and light chains into a cell, wherein the nucleotides encode light and chain variable domains which comprise framework regions produced according to the methods defined herein, and
expressing the nucleotides in a cell to produce the immunoglobulin.

A yet further aspect of the invention provides a method of producing an immunoglobulin which is substantially non-immunogenic when administered to a target species, the method comprising the steps of:

(1) comparing sequences of donor immunoglobulin heavy and/or light chain framework regions to a collection of sequences of target species immunoglobulin heavy and/or light chain framework regions;

(2) substituting any amino acid residues which are present at a specific position in the donor immunoglobulin framework region sequences, but not present at a corresponding position in the collection of target species immunoglobulin framework region sequences, with an amino acid residue which is present at a corresponding position in the collection of target species immunoglobulin framework region sequences;

(3) synthesizing a DNA segment encoding heavy and/or light chain variable regions, comprising CDRs from the donor immunoglobulin variable regions and the substituted framework regions, together with species-appropriate heavy and/or light chain constant domains;

(4) introducing the DNA segment into a cell; and

(5) expressing the DNA segment in the cell to produce the immunoglobulin.

In certain embodiments, the method further comprises the step of sequencing the donor immunoglobulin light chain and heavy chain variable region. In certain embodiments, the method further comprises the step of purifying the immunoglobulin.

In certain embodiments, the method further comprises the step of formulating the immunoglobulin in a pharmaceutically acceptable carrier or diluent.

A further aspect of the present invention provides a neutralising antibody or an antigen binding fragment thereof which is capable of specifically binding to human nerve growth factor (NGF) wherein the antibody or antigen binding fragment comprises a light chain variable region comprising, consisting of or consisting essentially of the amino acid sequence of SEQ ID NO:13 or an amino acid sequence which has an identity of at least 85%, 90%, 95% or 99% thereto and/or a heavy chain variable region comprising, consisting of or consisting essentially of the amino acid sequence of SEQ ID NO:14 or an amino acid sequence which has an identity of at least 85%, 90%, 95% or 99% thereto. In certain embodiments said identity is over a length of at least about 15 amino acids, preferably about 20 amino acids, more preferably about 25 amino acids.

The antibody may be prepared using a method of the invention.

In certain embodiments, the light chain comprises, consists of or consists essentially of the amino acid sequence of SEQ ID NO:25, or an amino acid sequence which has an identity of at least 85%, 90%, 95% or 99% thereto. In certain embodiments said identity is over a length of at least about 15 amino acids, preferably about 20 amino acids, more preferably about 25 amino acids.

In certain embodiments, the heavy chain comprises, consists of or consists essentially of the amino acid sequence of SEQ ID NO:24, or an amino acid sequence which has a sequence identity of at least 85%, 90%, 95% or 99% thereto. In certain embodiments said identity is over a length of at least about 15 amino acids, preferably about 20 amino acids, more preferably about 25 amino acids.

The inventor has further defined a series of framework regions (FR) which can be combined with complementarity determining regions (CDRs) to form humanised heavy and light chain variable domains. Each of the human heavy and light chain domains has 4 framework regions, designated FR1, FR2, FR3 and FR4.

Accordingly, also provided is a neutralising antibody or an antigen binding fragment thereof which is capable of specifically binding to human nerve growth factor (NGF) wherein the antibody or antigen binding fragment comprises a light chain variable region comprising at least one of:
an FR1 framework region consisting of or comprising the amino acid sequence of SEQ ID NO:60,
an FR2 framework region consisting of or comprising the amino acid sequence of SEQ ID NO:61,
an FR3 framework region consisting of or comprising the amino acid sequence of SEQ ID NO:62, and
an FR4 framework region consisting of or comprising the amino acid sequence of SEQ ID NO:63,

and/or a heavy chain variable region comprising at least one of:
an FR1 framework region consisting of or comprising the amino acid sequence of SEQ ID NO:64,
an FR2 framework region consisting of or comprising the amino acid sequence of SEQ ID NO:65,
an FR3 framework region consisting of or comprising the amino acid sequence of SEQ ID NO:66, and
an FR4 framework region consisting of or comprising the amino acid sequence of SEQ ID NO:67.

In certain embodiments, the light chain comprises all of light chain FR1, FR2, FR3 and FR4 and/or the heavy chain comprises all of heavy chain FR1, FR2, FR3 and FR4.

In certain embodiments, the antibody or binding fragment of the above aspects of the invention specifically binds to human NGF with a binding affinity having an equilibrium dissociation constant (K_{D}) of 1×10⁻⁸ or less.

In certain embodiments, the antibody or binding fragment of the above aspects of the invention inhibits the ability of human NGF to bind to the p75 or the TrkA human NGF receptors.

Preferably, the antibody or binding fragment of the above aspects of the invention is not immunogenic in humans.

Typically the antibody of the above aspects of the invention comprises light chain and/or heavy chain constant domains derived from an immunoglobulin derived from a human.

In certain embodiments, the binding fragment of the above aspects of the invention is selected from the group consisting of a single chain Fv (scFv) antibody fragment, a Fab antibody fragment, a Fab′ antibody fragment and a F(ab′)₂ antibody fragment.

In various further aspects, the present invention extends to an isolated nucleic acid which encodes the antibody or antigen binding fragments of the invention.

Accordingly, a yet further aspect of the invention provides an isolated nucleic acid that encodes an antibody or antigen binding fragment according to any of the foregoing aspects of the invention.

In certain embodiments, the polynucleotide encodes the light chain variable domain of an anti-NGF antibody or antigen binding fragment having the amino acid sequence of SEQ ID NO:13, or an amino acid sequence which has an identity of at least 85%, 90%, 95% or 99% thereto.

Also provided is an isolated nucleic acid that encodes the light chain of an anti-NGF antibody or antigen binding fragment having the amino acid sequence of SEQ ID NO:25, or an amino acid sequence which has an identity of at least 85%, 90%, 95% or 99% thereto.

In certain embodiments, the polynucleotide encodes the heavy chain variable domain of an anti-NGF antibody or antigen binding fragment having the amino acid sequence of SEQ ID NO:14, or an amino acid sequence which has an identity of at least 85%, 90%, 95% or 99% thereto.

Also provided is an isolated nucleic acid that encodes the heavy chain of an anti-NGF antibody or antigen binding fragment having the amino acid sequence of SEQ ID NO:24, or an amino acid sequence which has an identity of at least 85%, 90%, 95% or 99% thereto.

In certain embodiments, the isolated nucleic acid further comprises a nucleic acid encoding one or more regulatory sequences operably linked thereto.

In a further aspect there is provided an expression vector comprising a polynucleotide encoding a heavy and/or light chain variable domain or a heavy and/or light chain of the invention. In certain embodiments the expression vector further comprises one or more regulatory sequences. In certain embodiments the vector is a plasmid or a retroviral vector.

A yet further aspect provides a host cell incorporating the expression vector of the foregoing aspect of the invention. A further aspect of the invention provides a host cell which produces the antibody of any of the foregoing aspects of the invention.

A yet further aspect of the invention provides a method for producing a humanised NGF neutralising antibody, the method comprising the step of culturing the host cell of the foregoing aspect of the invention to allow the cell to express the humanised NGF neutralising antibody.

A yet further aspect of the present invention provides a method of producing an NGF neutralising antibody according to the invention comprising the steps of expressing one or more of the polynucleotides/nucleic acids or vectors of the foregoing aspects of the invention which express the light and/or heavy chains of the antibodies of the invention in a suitable host cell, recovering the expressed polypeptides, which may be expressed together in a host cell, or separately in different host cells, and isolating antibodies.

In certain embodiments, there is provided an antibody or binding fragment of the invention and at least one pharmaceutically acceptable diluent or carrier.

A yet further aspect of the present invention provides use of the antibody or binding fragment, nucleic acid, pharmaceutical composition or expression vector of the above aspects of the invention in the preparation of a medicament for the treatment or prevention of disease, such as arthritis, or for the treatment, prevention of amelioration of pain, such as pain associated with disease (e.g. neuropathic pain, post-operative pain, chronic pain, oncologic pain, etc). In various further aspects, the present invention extends to the use of the antibody or binding fragment of the above aspects of the invention in therapeutic and diagnostic methods.

A yet further aspect of the invention relates to the administration of the antibody or binding fragment, nucleic acid, pharmaceutical composition or expression vector of the above aspects of the invention to a subject, in particular a mammalian subject, for the treatment or prevention of disease (e.g. arthritis) or pain.

In certain embodiments, the disease is a condition caused by, associated with or resulting in increased sensitivity to nerve growth factor (NGF). In certain embodiments, the disease relates to a tumour induced to proliferate by NGF (e.g, an osteosarcoma).

In certain embodiments, the foregoing methods of the invention further comprise the step of co-administering at least one further agent which may enhance and/or complement the effectiveness of the anti-NGF antibody of the invention. For example, the antibody or antigen binding fragment thereof may be co-administered along with at least one analgesic, NSAID, opioid, corticosteroid, steroid, hyaluronan or hyaluronic acid.

In a yet further aspect there is provided a cell line, or a derivative or progeny cell thereof, that produces anti-human NGF neutralising monoclonal antibodies, or fragments thereof according to the invention.

A yet further aspect of the present invention provides a kit for the treatment of pain in humans, or for the treatment of a condition associated with pain, or for the treatment, amelioration or inhibition of pain associated with osteoarthritis, rheumatoid arthritis and inflammation, comprising an anti-NGF antibody according to any of the foregoing aspects of the invention and instructions for use of the same.

A yet further aspect of the present invention provides a diagnostic kit for the detection of an anti-human NGF monoclonal antibody in fluids in vitro, ex vivo and in vivo, for use in determining the concentration of said antibody. The kit may comprise any of the antibodies of the invention or a binding fragment thereof. The kit may comprise instructions for use of same.

A further aspect of the present invention provides a neutralising antibody or an antigen binding fragment thereof which is capable of specifically binding to canine tumour necrosis factor (TNF) wherein the antibody or antigen binding fragment comprises a light chain variable region comprising, consisting of or consisting essentially of the amino acid sequence of SEQ ID NO:71 or an amino acid sequence which has an identity of at least 85%, 90%, 95% or 99% thereto and/or a heavy chain variable region comprising, consisting of or consisting essentially of the amino acid sequence of SEQ ID NO:16 or an amino acid sequence which has an identity of at least 85%, 90%, 95% or 99% thereto. In certain embodiments said identity is over a length of at least about 15 amino acids, preferably about 20 amino acids, more preferably about 25 amino acids.

The antibody may be prepared using a method of the invention. Typically the antibody of the above aspects of the invention comprises light chain and/or heavy chain constant domains derived from an immunoglobulin derived from a canine. In certain embodiments, the heavy chain comprises, consists of or consists essentially of the amino acid sequence of SEQ ID NO:18, 19, 20 or 21, or an amino acid sequence which has a sequence identity of at least 85%, 90%, 95% or 99% thereto. In certain embodiments said identity is over a length of at least about 15 amino acids, preferably about 20 amino acids, more preferably about 25 amino acids.

In certain embodiments, the antibody or binding fragment of the above aspects of the invention specifically binds to canine TNF with a binding affinity having an equilibrium dissociation constant (K_{D}) of 1×10⁻⁸ or less. Preferably, the antibody or binding fragment of the above aspects of the invention is not immunogenic in canines.

In certain embodiments, the binding fragment of the above aspects of the invention is selected from the group consisting of a single chain Fv (scFv) antibody fragment, a Fab antibody fragment, a Fab′ antibody fragment and a F(ab′)₂ antibody fragment.

In various further aspects, the present invention extends to an isolated nucleic acid which encodes the antibody or antigen binding fragments of the invention.

Accordingly, a yet further aspect of the invention provides an isolated nucleic acid that encodes an antibody or antigen binding fragment according to any of the foregoing aspects of the invention.

In certain embodiments, the polynucleotide encodes the light chain variable domain of an anti-TNF antibody or antigen binding fragment having the amino acid sequence of SEQ ID NO:71, or an amino acid sequence which has an identity of at least 85%, 90%, 95% or 99% thereto.

In certain embodiments, the isolated nucleic acid further comprises a nucleic acid encoding one or more regulatory sequences operably linked thereto. In a further aspect there is provided an expression vector comprising a polynucleotide encoding a heavy and/or light chain variable domain or a heavy and/or light chain of the invention. In certain embodiments the expression vector further comprises one or more regulatory sequences. In certain embodiments the vector is a plasmid or a retroviral vector. A yet further aspect provides a host cell incorporating the expression vector of the foregoing aspect of the invention. A further aspect of the invention provides a host cell which produces the antibody of any of the foregoing aspects of the invention.

A yet further aspect of the invention provides a method for producing a caninised TNF neutralising antibody, the method comprising the step of culturing the host cell of the foregoing aspect of the invention to allow the cell to express the caninised TNF neutralising antibody.

A yet further aspect of the present invention provides a method of producing an TNF neutralising antibody according to the invention comprising the steps of expressing one or more of the polynucleotides/nucleic acids or vectors of the foregoing aspects of the invention which express the light and/or heavy chains of the antibodies of the invention in a suitable host cell, recovering the expressed polypeptides, which may be expressed together in a host cell, or separately in different host cells, and isolating antibodies.

In certain embodiments, there is provided an antibody or binding fragment of the invention and at least one pharmaceutically acceptable diluent or carrier.

A yet further aspect of the present invention provides use of the antibody or binding fragment, nucleic acid, pharmaceutical composition or expression vector of the above aspects of the invention in the preparation of a medicament for the treatment or prevention of disease, in particular any condition caused by, associated with or resulting in increased expression of canine TNF or increased sensitivity to TNF in a canine. In various further aspects, the present invention extends to the use of the antibody or binding fragment of the above aspects of the invention in therapeutic and diagnostic methods.

A yet further aspect of the invention relates to the administration of the antibody or binding fragment, nucleic acid, pharmaceutical composition or expression vector of the above aspects of the invention to a canine, for the treatment or prevention of disease.

In a yet further aspect there is provided a cell line, or a derivative or progeny cell thereof, that produces anti-canine TNF neutralising monoclonal antibodies, or fragments thereof according to the invention.

A further aspect of the present invention provides a neutralising antibody or an antigen binding fragment thereof which is capable of specifically binding to canine nerve growth factor (NGF) wherein the antibody or antigen binding fragment comprises a light chain variable region comprising, consisting of or consisting essentially of the amino acid sequence of SEQ ID NO:1 or an amino acid sequence which has an identity of at least 85%, 90%, 95% or 99% thereto and/or a heavy chain variable region comprising, consisting of or consisting essentially of the amino acid sequence of SEQ ID NO:69 or an amino acid sequence which has an identity of at least 85%, 90%, 95% or 99% thereto. In certain embodiments said identity is over a length of at least about 15 amino acids, preferably about 20 amino acids, more preferably about 25 amino acids.

The antibody may be prepared using a method of the invention.

In certain embodiments, the light chain comprises, consists of or consists essentially of the amino acid sequence of SEQ ID NO:7, or an amino acid sequence which has an identity of at least 85%, 90%, 95% or 99% thereto. In certain embodiments said identity is over a length of at least about 15 amino acids, preferably about 20 amino acids, more preferably about 25 amino acids.

In certain embodiments, the heavy chain comprises, consists of or consists essentially of the amino acid sequence of SEQ ID NO:70, or an amino acid sequence which has a sequence identity of at least 85%, 90%, 95% or 99% thereto. In certain embodiments said identity is over a length of at least about 15 amino acids, preferably about 20 amino acids, more preferably about 25 amino acids.

In certain embodiments, the antibody or binding fragment of the above aspects of the invention specifically binds to canine NGF with a binding affinity having an equilibrium dissociation constant (K_{D}) of 1×10⁻⁸ or less. In certain embodiments, the antibody or binding fragment of the above aspects of the invention inhibits the ability of canine NGF to bind to the p75 or the TrkA canine NGF receptors. Preferably, the antibody or binding fragment of the above aspects of the invention is not immunogenic in canines.

Typically the antibody of the above aspects of the invention comprises light chain and/or heavy chain constant domains derived from an immunoglobulin derived from a canine.

In certain embodiments, the binding fragment of the above aspects of the invention is selected from the group consisting of a single chain Fv (scFv) antibody fragment, a Fab antibody fragment, a Fab′ antibody fragment and a F(ab′)₂ antibody fragment.

In various further aspects, the present invention extends to an isolated nucleic acid which encodes the antibody or antigen binding fragments of the invention.

Accordingly, a yet further aspect of the invention provides an isolated nucleic acid that encodes an antibody or antigen binding fragment according to any of the foregoing aspects of the invention.

In certain embodiments, the polynucleotide encodes the light chain variable domain of an anti-NGF antibody or antigen binding fragment having the amino acid sequence of SEQ ID NO:1, or an amino acid sequence which has an identity of at least 85%, 90%, 95% or 99% thereto.

Also provided is an isolated nucleic acid that encodes the light chain of an anti-NGF antibody or antigen binding fragment having the amino acid sequence of SEQ ID NO:7, or an amino acid sequence which has an identity of at least 85%, 90%, 95% or 99% thereto.

In certain embodiments, the polynucleotide encodes the heavy chain variable domain of an anti-NGF antibody or antigen binding fragment having the amino acid sequence of SEQ ID NO:69, or an amino acid sequence which has an identity of at least 85%, 90%, 95% or 99% thereto.

Also provided is an isolated nucleic acid that encodes the heavy chain of an anti-NGF antibody or antigen binding fragment having the amino acid sequence of SEQ ID NO:70, or an amino acid sequence which has an identity of at least 85%, 90%, 95% or 99% thereto.

In certain embodiments, the isolated nucleic acid further comprises a nucleic acid encoding one or more regulatory sequences operably linked thereto.

In a further aspect there is provided an expression vector comprising a polynucleotide encoding a heavy and/or light chain variable domain or a heavy and/or light chain of the invention. In certain embodiments the expression vector further comprises one or more regulatory sequences. In certain embodiments the vector is a plasmid or a retroviral vector.

A yet further aspect provides a host cell incorporating the expression vector of the foregoing aspect of the invention. A further aspect of the invention provides a host cell which produces the antibody of any of the foregoing aspects of the invention.

A yet further aspect of the invention provides a method for producing a caninised NGF neutralising antibody, the method comprising the step of culturing the host cell of the foregoing aspect of the invention to allow the cell to express the caninised NGF neutralising antibody.

A yet further aspect of the present invention provides a method of producing an NGF neutralising antibody according to the invention comprising the steps of expressing one or more of the polynucleotides/nucleic acids or vectors of the foregoing aspects of the invention which express the light and/or heavy chains of the antibodies of the invention in a suitable host cell, recovering the expressed polypeptides, which may be expressed together in a host cell, or separately in different host cells, and isolating antibodies.

In certain embodiments, there is provided an antibody or binding fragment of the invention and at least one pharmaceutically acceptable diluent or carrier.

A yet further aspect of the present invention provides use of the antibody or binding fragment, nucleic acid, pharmaceutical composition or expression vector of the above aspects of the invention in the preparation of a medicament for the treatment or prevention of disease, such as arthritis, or for the treatment, prevention of amelioration of pain, such as pain associated with disease (e.g. neuropathic pain, post-operative pain, chronic pain, oncologic pain, etc) in a canine. In various further aspects, the present invention extends to the use of the antibody or binding fragment of the above aspects of the invention in therapeutic and diagnostic methods.

A yet further aspect of the invention relates to the administration of the antibody or binding fragment, nucleic acid, pharmaceutical composition or expression vector of the above aspects of the invention to a canine for the treatment or prevention of disease (e.g. arthritis) or pain.

In certain embodiments, the disease is a condition caused by, associated with or resulting in increased sensitivity to nerve growth factor (NGF). In certain embodiments, the disease relates to a tumour induced to proliferate by NGF (e,g, an osteosarcoma).

In certain embodiments, the foregoing methods of the invention further comprise the step of co-administering at least one further agent which may enhance and/or complement the effectiveness of the anti-NGF antibody of the invention. For example, the antibody or antigen binding fragment thereof may be co-administered along with at least one analgesic, NSAID, opioid, corticosteroid, steroid, hyaluronan or hyaluronic acid.

In a yet further aspect there is provided a cell line, or a derivative or progeny cell thereof, that produces anti-canine NGF neutralising monoclonal antibodies, or fragments thereof according to the invention.

A yet further aspect of the present invention provides a kit for the treatment of pain in canines, or for the treatment of a condition associated with pain, or for the treatment, amelioration or inhibition of pain associated with osteoarthritis, rheumatoid arthritis and inflammation, comprising an anti-NGF antibody according to any of the foregoing aspects of the invention and instructions for use of the same.

A yet further aspect of the present invention provides a diagnostic kit for the detection of an anti-canine NGF monoclonal antibody in fluids in vitro, ex vivo and in vivo, for use in determining the concentration of said antibody. The kit may comprise any of the antibodies of the invention or a binding fragment thereof. The kit may comprise instructions for use of same.

### BRIEF DESCRIPTION OF THE FIGURES

FIG. 1 shows an alignment of framework region sequences FR1 to FR4 of the light chain of the rat αD11 antibody with canine, feline and equine species specific versions (SEQ ID NO:28 to 43).

FIG. 2 shows an alignment of framework region sequences FR1 to FR4 of the heavy chain of the rat αD11 antibody with canine, feline and equine species specific versions (SEQ ID NO:44 to 59).

FIG. 3 shows amino acid sequences of the heavy (SEQ ID NO:2) and light (SEQ ID NO:1) chain variable domains of a canine version of αD11 anti-NGF MAb and of the complete light (SEQ ID NO:7) and heavy chain (SEQ ID NO:8).

FIG. 4 shows amino acid sequences of the heavy (SEQ ID NO:4) and light (SEQ ID NO:3) chain variable domains of a feline version of αD11 anti-NGF MAb and of the complete light (SEQ ID NO:9) and heavy (SEQ ID NO:10) chains.

FIG. 5 shows amino acid sequences of the heavy (SEQ ID NO:6) and light (SEQ ID NO:5) chain variable domains of an equine version of αD11 anti-NGF MAb and of the complete light (SEQ ID NO:11) and heavy (SEQ ID NO:12) chains.

FIG. 6 shows a gel with the expression of canine, feline and equine speciesised versions of the αD11 MAb.

FIG. 7A is a graph showing that expressed and purified canine MAbs to canine NGF are biologically active. FIG. 7B is a graph showing that expressed and purified feline MAbs to feline NGF are biologically active, while FIG. 7C is a graph showing that expressed and purified equine MAbs to equine NGF are biologically active. FIG. 7D is a graph comparing the ability of canine, feline and equine MAbs to neutralise NGF biological activity.

FIG. 8A is an amino acid alignment showing a comparison of light chain framework region modifications between a known humanised version of the rat alpha D11 antibody (Pavone et al, WO 06/131951) and a novel humanised variant of αD11 (New Hu—SEQ ID NO:60-63). FIG. 8B is a comparison of heavy chain framework region modifications between the known humanised version of the rat alpha D11 antibody (Pavone et al, WO 06/131951) and the novel humanised variant of αD11 (New Hu—SEQ ID NO:64-67).

FIG. 9A shows the light chain (SEQ ID NO:13) and heavy chain (SEQ ID NO:14) variable domain amino acid sequences of the novel humanised alpha D11 antibody from FIG. 8-CDRs are underlined. FIG. 9B shows complete heavy and light chains (SEQ ID NO:24 and 25) designed using the light chain and heavy chain variable domains shown in FIG. 9A. FIG. 9C shows an ELISA assay comparing an antibody made from the sequences in FIG. 9B with an antibody designed by CDR grafting, as previously described by Pavone and colleagues. FIG. 9D shows inhibition of NGF proliferation of TF-1 cells by the two humanised variants of alpha D11 monoclonal antibodies used in FIG. 9C.

FIG. 10 shows light chain (SEQ ID NO:15) and heavy chain (SEQ ID NO:16) variable domain amino acid sequences of a caninised anti-TNF antibody based on the human MAb D2E7 (Salfield et al., U.S. Pat. No. 6,090,382).

FIG. 11 shows the kappa light chain amino acid sequence (SEQ ID NO:17) of a caninised anti-TNF antibody.

FIG. 12 shows the heavy chain amino acid sequences of 4 subtypes (heavy chain type A, B, C and D) of a caninised anti-TNF antibody.

FIG. 13 shows the complete light chain (SEQ ID NO:22) and heavy chain (SEQ ID NO:23) amino acid sequences of a chimeric human-canine anti-TNF antibody.

FIG. 14A shows the results of co-expressed caninised (Ca) and chimeric (Ch) anti-TNF antibodies purified using Protein A and analysed by SDS-PAGE, while FIG. 14B shows the results of an ELISA showing binding of expressed recombinant proteins to canine TNF-alpha. Results with various dilutions of antibodies from 5 μg/ml to 0.05 μg/ml are shown.

FIG. 15 shows inhibition of canine TNF bioactivity using 293-HEK cells transfected with the NF-kB-EGFP reporter construct pTRH1. These cells respond to canine TNF by fluorescence. Both the caninised (FIG. 15A) and chimeric (FIG. 15B) MAbs inhibited TNF-induced fluorescence equally well, as quantified in FIG. 15C.

FIG. 16 shows a comparison to a further caninised MAb based on anti-TNF MAb clone 148 expressed in CHO cells and purified using Protein A chromatography (Panel A, left lane). The MAb was tested for binding to human TNF (Panel B) and canine TNF (Panel C) in comparison to the caninised (Ca) and chimeric (Ch) D2E7 based MAbs from FIG. 14 (background negative control binding is shown by the arrows).

FIG. 17 shows the heavy chain (SEQ ID NO:26-ca148-HCB) and light chain (SEQ ID NO:27-ca148-kLC) of caninised MAb 148.

FIG. 18 shows that anti-canine NGF monoclonal antibodies prepared by a method of the present invention reduce inflammatory pain in dogs.

### DETAILED DESCRIPTION OF THE INVENTION

The present invention extends to novel methods for designing and making a variant of a donor antibody which has reduced immunogenicity when administered to a species which is different to that from which the antibody is derived, while at the same time maintaining (i.e. not decreasing) the binding affinity, avidity or specificity of the antibody for the target ligand. In particular the sequence of the framework regions of the antibody or an antibody binding fragment are assessed and modified to remove residues which are not found at a corresponding position in a pool of antibodies derived from the species to which the antibody is to be administered.

In particular, the methods of the invention modify the framework of a donor (parental) antibody so that it is optimally compatible with the intended recipient where the intended recipient is a species other than the donor.

Hitherto in the art, the most commonly used approach to de-immunising an antibody has been to select a compatible framework from germline sequence of a recipient species and to graft the CDR regions into this framework. The problem with this approach is that the selected framework is rarely a perfect match for the CDRs and, as a result, binding affinity to the intended target epitope is reduced. Affinity maturation is then required which results in the introduction of amino acids that are sometimes foreign to the recipient at the positions at which they are introduced. The solution is the procedure of this invention where modification of the donor framework is only undertaken where an amino acid is foreign to the recipient at a specific position. The replacement is chosen from the list of options assembled as part of this invention. As a result, which is the crux of the invention, there is essentially no structural modification of the donor framework and hence essentially no distortion of the conformation of the CDRs, but at the same time there are no “non-self” amino acids within the framework and the resultant immunoglobulin lacks framework epitopes that are foreign in the target species. The methodology can be used to modify an immunoglobulin for administration to any desired target species.

In the most expansive study of immunoglobulin sequence variety to date, Glanville and colleagues (2009) studied the amino acid composition of nearly 100,000 heavy and light chain cDNAs amplified from naive IgM of 654 human donors. 95% of sequences differed from germline by as many as 30 mutations each. CDRs 1 and 2 which mutate only via somatic mutation were demonstrated to be unaltered from germline DNA only 17% of the time and 78% of CDR 1 and 2 sequences had between 1 and 6 amino acid differences. This study did not describe the mutation of non-CDR framework residues, described in the Tables shown herein. Given that somatic hypermutation is via an error prone DNA replication mechanism, the mutation rate per se is unlikely to be different between that observed for CDR 1 or 2 and the rest of the framework sequences and, furthermore, profound secondary selection following somatic hypermutation in the antigen-experienced repertoire during maturation of the immune response would permit further amino acid diversity in framework regions. For these reasons, the diversity observed in the collections of human, canine, equine and feline IgG sequences described herein are likely to be representative of circulating IgG sequences. Surprisingly, there is considerable overlap between the amino acids encoded at homologous “Kabat” framework positions of immunoglobulins of each species post-somatic hypermutation and, consequently, this reduces the changes necessary to convert from one species to another according to the method of the present invention.

As an example of this invention and following extensive experimentation, the inventor has taken the D2E7 anti-human TNF antibody and the αD11 rat anti-mouse NGF antibody, which were not known to bind to canine TNF alpha or canine NGF, and has surprisingly used these as a basis to produce non-immunogenic antibodies suitable for use in canines. The resulting non-immunogenic antibodies, which are not produced using standard CDR grafting techniques, are shown to exhibit high affinity binding to canine TNF and NGF respectively. Furthermore, the antibodies have been designed so that the framework and constant regions incorporate only residues present in canine IgG molecules so that when administered to a canine, xenoantibodies are unlikely to be produced there against. Accordingly, the caninised antibodies of the invention are suitable for long-term administration for the treatment of diseases in canines. Similarly, the felinised, humanised and equinised NGF antibodies of the invention are suitable for long-term administration for the treatment of diseases in felines, humans and equines respectively.

The process of generating the heavy and light chain variable domains for the antibodies of the invention which has been employed by the inventor results in the replacement of specific donor amino acid residues known to be foreign to the target (e.g. canines) at that position with a target (e.g. canine) residue which, based on the inventor's analysis, will retain the conformation of the CDR regions and therefore maintain binding specificity and avidity, while reducing the presence of immunogenic epitopes which may result in neutralising antibodies being generated against the antibody if it were to be administered to the target (e.g. canines) in an unaltered form. Specifically, the method of preparing antibodies of the invention (known as PETisation) comprises assessing the sequence of the framework regions of a donor (e.g. human) antibody for suitability for administering to a target (e.g. canine) by comparing the sequence of the framework regions of the donor antibody with the sequence of an antibody or a pool of antibodies derived from the target (e.g. canine). Although the comparison may be between the donor sequence and a single member of the target sequence, it will be obvious that comparison with a pool of target sequences is preferred because this will expand the number of natural options at each Kabat position in the target species. Not only will this increase the chance of a “match” between the donor and the target, but it will also expand the options for replacement where a match does not exist. As a result, a replacement with characteristics as close as possible to the donor will be able to be chosen. Where the donor sequence and the canine sequence differ at any Kabat number or corresponding position, the donor sequence is modified to substitute the amino acid residue in question with an amino acid residue which is known to be natural at that position in the target (e.g. canines).

Where substitution of an amino acid residue present in a donor immunoglobulin framework region is required, typically this is undertaken using the principle of conservative substitution wherein an amino acid residue is replaced with an amino acid residue which is natural at that Kabat position in the target (e.g. a canine) and is as closely related as possible in size, charge and hydrophobicity to the amino acid being substituted in the donor sequence. The intention is to choose a replacement which would cause no, or at least only minimum, perturbation or disruption to the three-dimensional structure of the donor antibody. In certain situations, there will be no clear option and each choice will have benefits and downsides. A final decision may require three-dimensional modelling or even expression of various alternative sequences. However, generally, a clear preference will be available. As a result of this procedure, a change in the donor sequence is only made when that residue would be foreign in the target and the replacement amino acid is as closely related as possible to that which it replaces. Thus, the creation of foreign epitopes is avoided, but the overall three-dimensional structure is preserved and as a result, affinity and specificity are also preserved. Exemplary examples of this aspect of the invention include:
light chain sequences SEQ ID NO: 1, 3, 5, 7, 9, 11, 13, 15, 17, 25, 27 and 71.
heavy chain sequences SEQ ID NO: 2, 4, 6, 8, 10, 12, 14, 16, 18, 19, 20, 21, 24, 26, 68, 69 and 70.

### Antibody Production

The antibodies and binding members of the invention may be produced wholly or partly by chemical synthesis. For example, the antibodies and binding members of the invention can be prepared by techniques which are well known to the person skilled in the art, such as standard liquid peptide synthesis, or by solid-phase peptide synthesis methods. Alternatively, the antibodies and binding members may be prepared in solution using liquid phase peptide synthesis techniques, or further by a combination of solid-phase, liquid phase and solution chemistry.

The present invention further extends to the production of the antibodies or binding members of the invention by expression of a nucleic acid which encodes at least one amino acid which comprises an antibody of the invention in a suitable expression system, such that a desired peptide or polypeptide can be encoded. For example, a first nucleic acid encoding the amino acid light chain and a second nucleic acid encoding an amino acid heavy chain can be expressed to provide an antibody of the present invention.

Accordingly, in certain further aspects of the invention, there is provided nucleic acids encoding amino acid sequences which form the antibodies or binding members of the present invention.

Typically, nucleic acids encoding the amino acid sequences which form antibodies or binding members of the present invention can be provided in an isolated or purified form, or provided in a form which is substantially free of material which can be naturally associated with it, with the exception of one or more regulatory sequences. Nucleic acids which expresses an antibody or binding member of the invention may be wholly or partially synthetic and may include, but are not limited to, DNA, cDNA and RNA.

Nucleic acid sequences encoding the antibodies or binding members of the invention can be readily prepared by the skilled person using techniques which are well known to those skilled in the art, such as those described in Sambrook et al. “Molecular Cloning”, A laboratory manual, Cold Spring Harbor Laboratory Press, Volumes 1-3, 2001 (ISBN-0879695773), and Ausubel et al. Short Protocols in Molecular Biology. John Wiley and Sons, 4^{th} Edition, 1999 (ISBN-0471250929). Said techniques include (i) the use of the polymerase chain reaction (PCR) to amplify samples of nucleic acid, (ii) chemical synthesis, or (iii) preparation of cDNA sequences. DNA encoding antibodies or binding members of the invention may be generated and used in any suitable way known to those skilled in the art, including taking encoding DNA, identifying suitable restriction enzyme recognition sites either side of the portion to be expressed, and cutting out said portion from the DNA. The excised portion may then be operably linked to a suitable promoter and expressed in a suitable expression system, such as a commercially available expression system. Alternatively, the relevant portions of DNA can be amplified by using suitable PCR primers. Modifications to the DNA sequences can be made by using site directed mutagenesis.

Nucleic acid sequences encoding the antibodies or binding members of the invention may be provided as constructs in the form of a plasmid, vector, transcription or expression cassette which comprises at least one nucleic acid as described above. The construct may be comprised within a recombinant host cell which comprises one or more constructs as above. Expression may conveniently be achieved by culturing, under appropriate conditions, recombinant host cells containing suitable nucleic acid sequences. Following expression, the antibody or antibody fragments may be isolated and/or purified using any suitable technique, then used as appropriate.

Systems for cloning and expression of a polypeptide in a variety of different host cells are well known. Suitable host cells include bacteria, mammalian cells, yeast, insect and baculovirus systems. Mammalian cell lines available in the art for expression of a heterologous polypeptide include Chinese hamster ovary (CHO) cells, HeLa cells, baby hamster kidney cells and NSO mouse myeloma cells. A common, preferred bacterial host is E. coli. The expression of antibodies and antibody fragments in prokaryotic cells such as E. coli is well established in the art. Expression in eukaryotic cells in culture is also available to those skilled in the art as an option for production of a binding member.

General techniques for the production of antibodies are well known to the person skilled in the field, with such methods being discussed in, for example, Kohler and Milstein (1975) Nature 256: 495-497; U.S. Pat. No. 4,376,110; Harlow and Lane, Antibodies: a Laboratory Manual, (1988) Cold Spring Harbor. Techniques for the preparation of recombinant antibody molecules are described in the above references and also in, for example, European Patent Number 0,368,684.

In certain embodiments of the invention, recombinant nucleic acids comprising an insert coding for a heavy chain variable domain and/or for a light chain variable domain of antibodies or binding members are employed. By definition, such nucleic acids comprise single stranded nucleic acids, double stranded nucleic acids consisting of said coding nucleic acids and of complementary nucleic acids thereto, or these complementary (single stranded) nucleic acids themselves.

Furthermore, nucleic acids encoding a heavy chain variable domain and/or a light chain variable domain of antibodies can be enzymatically or chemically synthesised nucleic acids having the authentic sequence coding for a naturally-occurring heavy chain variable domain and/or for the light chain variable domain, or a mutant thereof.

An antibody of the invention may be produced by recombinant means, not only directly, but also as a fusion polypeptide with a heterologous polypeptide, which is preferably a signal sequence or other polypeptide having a specific cleavage site at the N-terminus of the mature protein or polypeptide. The selected heterologous signal sequence preferably is one that is recognized and processed (i.e., cleaved by a signal peptidase) by the host cell. For prokaryotic host cells that do not recognize and process a native antibody signal sequence, the signal sequence is substituted by a prokaryotic signal sequence selected, for example, from the group of the alkaline phosphatase, penicillinase, lpp and heat-stable enterotoxin II leaders.

The term “isolated”, when used in reference to the antibodies of the invention, or to binding members derived therefrom, or polypeptides which encode the same, refers to the state in which said antibodies, binding members or nucleic acids (polynucleotides) are provided in an isolated and/or purified form, that is they have been separated, isolated or purified from their natural environment, and are provided in a substantially pure or homogeneous form, or, in the case of nucleic acid, free or substantially free of nucleic acid or genes of origin other than the sequence encoding a polypeptide with the required function. Accordingly, such isolated antibodies, binding members and isolated nucleic acids will be free or substantially free of material with which they are naturally associated, such as other polypeptides or nucleic acids with which they are found in their natural environment, or the environment in which they are prepared (e.g. cell culture) when such preparation is by recombinant DNA technology practised in vitro or in vivo.

Antibodies, binding members and nucleic acids may be formulated with diluents or adjuvants and still, for practical purposes, be considered as being provided in an isolated form. For example the antibodies and binding members can be mixed with gelatin or other carriers if used to coat microtitre plates for use in immunoassays, or will be mixed with pharmaceutically acceptable carriers or diluents when used in diagnosis or therapy. The antibodies or binding members may be glycosylated, either naturally or by systems of heterologous eukaryotic cells (e.g. CHO or NSO cells), or they may be (for example, if produced by expression in a prokaryotic cell) unglycosylated.

Heterogeneous preparations comprising antibodies of the invention also form part of the invention. For example, such preparations may be mixtures of antibodies with full-length heavy chains and heavy chains lacking the C-terminal lysine, with various degrees of glycosylation and/or with derivatized amino acids, such as cyclization of an N-terminal glutamic acid to form a pyroglutamic acid residue.

### DEFINITIONS

Unless otherwise defined, all technical and scientific terms used herein have the meaning commonly understood by a person who is skilled in the art in the field of the present invention. The meaning and scope of the terms should be clear, however, in the event of any ambiguity, definitions provided herein take precedent over any dictionary or extrinsic definition.

Throughout the specification, unless the context demands otherwise, the terms “comprise” or “include”, or variations such as “comprises” or “comprising”, “includes” or “including” will be understood to imply the inclusion of a stated integer or group of integers, but not the exclusion of any other integer or group of integers.

As used herein, terms such as “a”, “an” and “the” include singular and plural referents unless the context clearly demands otherwise. Thus, for example, reference to “an active agent” or “a pharmacologically active agent” includes a single active agent as well as two or more different active agents in combination, while references to “a carrier” includes mixtures of two or more carriers as well as a single carrier, and the like. Further, unless otherwise required by context, singular terms shall include pluralities and plural terms shall include the singular.

The term “corresponding amino acid” means an amino acid residue which is found at an identical position (i.e., they lie across from each other) when two or more amino acid sequences are aligned to allow for maximum sequence identity between the sequences. Amino acid residues at corresponding positions have the same Kabat numbering. In particular, amino acid sequences of framework regions of different antibodies may be aligned or the amino acid sequence of a framework region sequence of one antibody may be compared to a pool of positional specific framework region amino acid residues derived from a plurality of immunoglobulins of a particular species. Methods for aligning and numbering antibody sequences are well known to the person skilled in the art and are disclosed in Kabat et al. (Kabat, E. A., Wu, T. T., Perry, H., Gottesman, K. and Foeller, C. (1991) Sequences of Proteins of Immunological Interest, Fifth Edition. NIH Publication No. 91-3242), the contents of which are incorporated by reference.

The term “complementarity determining region (CDR)”, as used herein, refers to amino acid sequences which together define the binding affinity and specificity of the natural Fv region of a native immunoglobulin binding site as delineated by Kabat et al. (Kabat, E. A., Wu, T. T., Perry, H., Gottesman, K. and Foeller, C. (1991) Sequences of Proteins of Immunological Interest, Fifth Edition. NIH Publication No. 91-3242). The term “framework region (FR)”, as used herein, refers to amino acid sequences interposed between CDRs. These portions of the antibody serve to hold the CDRs in appropriate orientation (allows for CDRs to bind antigen).

The term “constant region (CR)” as used herein, refers to the portion of the antibody molecule which confers effector functions. In the present invention, constant regions typically mean constant regions of the target species, that is, that the constant regions of the subject speciesised antibodies are derived from immunoglobulins of the target species. For example, in canine antibodies the heavy chain constant region can be selected from any of four isotypes A, B, C or D.

The term “chimeric antibody” as used herein refers to an antibody containing sequences derived from two different antibodies, which typically are of different species. Most typically, chimeric antibodies comprise variable domains derived from a donor species which bind specifically to a target epitope and constant domains derived from antibodies obtained from the target species to whom the antibody is to be administered.

The term “immunogenicity” as used herein refers to a measure of the ability of a targeting protein or therapeutic moiety to elicit an immune response (humoral or cellular) when administered to a recipient. The present invention is concerned with the immunogenicity of the subject speciesised antibodies. Preferably the antibodies of the present invention have no immunogenicity, that is, that no neutralising antibodies will be raised against them when administered to a target species.

The term “consists essentially of” or “consisting essentially of” as used herein means that a polypeptide may have additional features or elements beyond those described provided that such additional features or elements do not materially affect the ability of the antibody or antibody fragment to have binding specificity to the desired target. That is, the antibody or antibody fragments comprising the polypeptides may have additional features or elements that do not interfere with the ability of the antibody or antibody fragments to bind to the desired target and antagonise its functional activity. Such modifications may be introduced into the amino acid sequence in order to reduce the immunogenicity of the antibody. For example, a polypeptide consisting essentially of a specified sequence may contain one, two, three, four, five or more additional, deleted or substituted amino acids at either end or at both ends of the sequence provided that these amino acids do not interfere with, inhibit, block or interrupt the role of the antibody or fragment in binding to the desired target and sequestering its biological function. Similarly, a polypeptide molecule which contributes to the antagonistic antibodies of the invention may be chemically modified with one or more functional groups provided that such functional groups do not interfere with the ability of the antibody or antibody fragment to bind to the desired target and antagonise its function.

The present invention will now be described with reference to the following examples which are provided for the purpose of illustration and are not intended to be construed as being limiting on the present invention.

### EXAMPLES

### Example 1

### Conversion of Murine Antibodies to Canine, Feline, Equine and Human Antibodies

Immunoglobulin gamma (IgG) variable domain heavy (VH) and light (VL) chain protein sequences of human, feline, canine and equine origin, derived from publicly available expressed cDNA sequence databases and publications, were aligned in groups according to species using the program ClustalW using the BLOSUM Cost Matrix and Gap open cost of 10 and Gap extend cost of 0.1. Poor quality sequences of low homology were deleted from the alignment to avoid spurious gaps in framework regions. Framework and CDR regions were identified according to the Kabat nomenclature and the amino acid residues at each Kabat framework region position were identified and tabulated according to light and heavy chain (Tables 1-8). Although the light chain table is constructed from a collection of kappa light chains, similar tables can be constructed from lambda light chains for use in converting lambda light chains from one species to another according to the methods described in this patent.

The conservation of sequence between the four species' IgG sequences at light chain framework positions Q6, C23, W35, P44, Y83, C85 and G98, and heavy chain framework positions G8, C22, W36, R38, D86, Y90, C92 and G106 suggest that there is minimal contamination of the pooled amino acid data set by simple nucleotide sequence errors in the starting data set. The composition of the pool of residues present at each framework region in Tables 1-8 is determined by the available data for each species. The determination of additional amino acid sequences for immunoglobulins derived from any of the analysed species could further diversify the composition of choices at any of the positions in the Tables. This is particularly true of feline derived variable light chains for which only a single example is currently available in the literature and for which the inventor has generated a new set by degenerate oligonucleotide primed polymerase chain reaction amplification of feline spleen tissue mRNA IgG light chain sequences, as shown in Tables 1-8. Nevertheless, as will be demonstrated below, the current tables can be used along with the methodology disclosed herein to produce antibodies which are suitable for administration to a number of different species.

The method according to the invention uses the information provided in Tables 1 to 8 to compare amino acid residues which are present at each position of the framework regions of the light and/or heavy chains of a donor immunoglobulin with a residue present in the pool of immunoglobulins derived from the target species. Should the amino acid residue present at a specific position of the donor framework region not be an amino acid which is present in that position in the pool of immunoglobulins derived from the target species, then the residue is substituted with an amino acid present in the pool as relevant to that position. When determining which amino acid should replace the substituted residue in the donor sequence, it is preferable to substitute the donor residue with the residue from the pool which is the nearest homologous residue. That is, the substitution is preferably a conservative substitution. If no homologous residue is available, then the consensus pool amino acid (i.e. the amino acid which is most commonly found at that position) of the target species may preferably be chosen for substitution.

In determining whether a substituted amino acid can be replaced with a conserved amino acid, an assessment may typically made of factors such as, but not limited to, (a) the structure of the polypeptide backbone in the area of the substitution, for example, as a sheet or helical conformation, (b) the charge or hydrophobicity of the molecule at the target site, and/or (c) the bulk of the side chain.

When considering whether a donor amino acid not present in the target pool can be conservatively substituted, it may be preferable to assess whether a homologous amino acid is present based on the amino acids being grouped together according to similarities in the properties of their side chains (A. L. Lehninger, in Biochemistry, 2^{nd} Ed., 73-75, Worth Publishers, New York (1975)). For example, the following groups can be determined: (1) non-polar: Ala (A), VaI (V), Leu (L), Ile (I), Pro (P), Phe (F), Trp (W), Met (M); (2) uncharged polar: GIy (G), Ser (S), Thr (T), Cys (C), Tyr (Y), Asn (N), Gln (O); (3) acidic: Asp (D), GIu (E); and (4) basic: Lys (K), Arg (R), His (H).

Alternatively, amino acid residues may be divided into groups based on common side-chain properties: (1) hydrophobic: Met, Ala, VaI, Leu, Ile; (2) neutral hydrophilic: Cys, Ser, Thr, Asn, GIn; (3) acidic: Asp, GIu; (4) basic: His, Lys, Arg; (5) residues that influence chain orientation: GIy, Pro; and (6) aromatic: Trp, Tyr, Phe.

Conservative substitutions will entail exchanging (substituting) a member of one of these classes for another member of that same class.

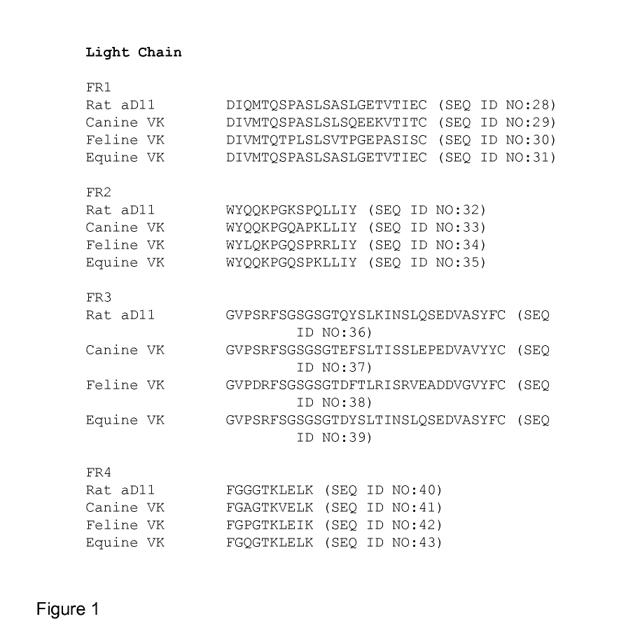

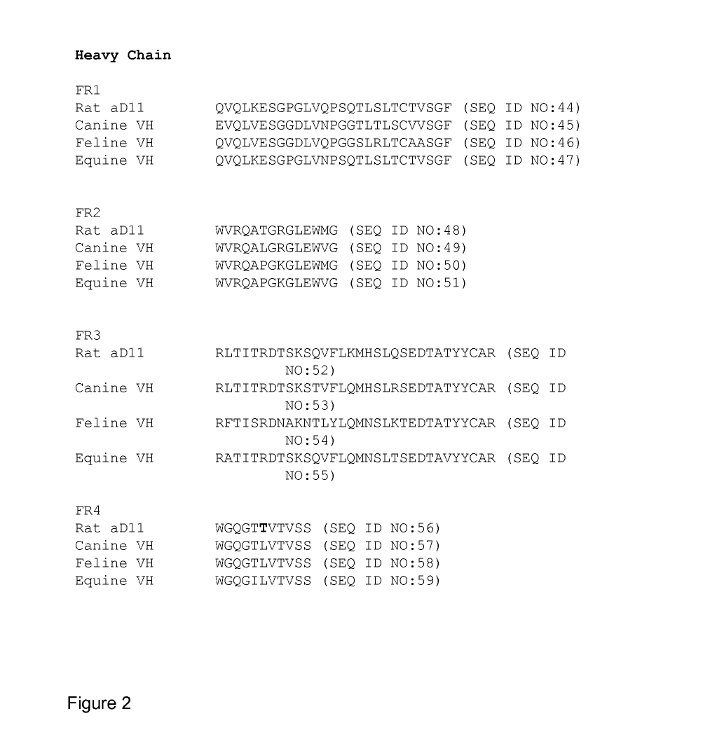

By way of example, donor immunoglobulin light and heavy chain variable domain sequences determined from a rat anti-mouse NGF monoclonal antibody (αD11) were aligned with these tables and PETised canine, equine and feline variants were designed and constructed for expression. The framework region residues selected for each species are shown in FIG. 1 and the differences from the donor rat sequence are shown in FIG. 2. As can be seen from FIGS. 1 and 2, the changes necessary to make canine, feline or equine versions of αD11 are different in number and type for each species.

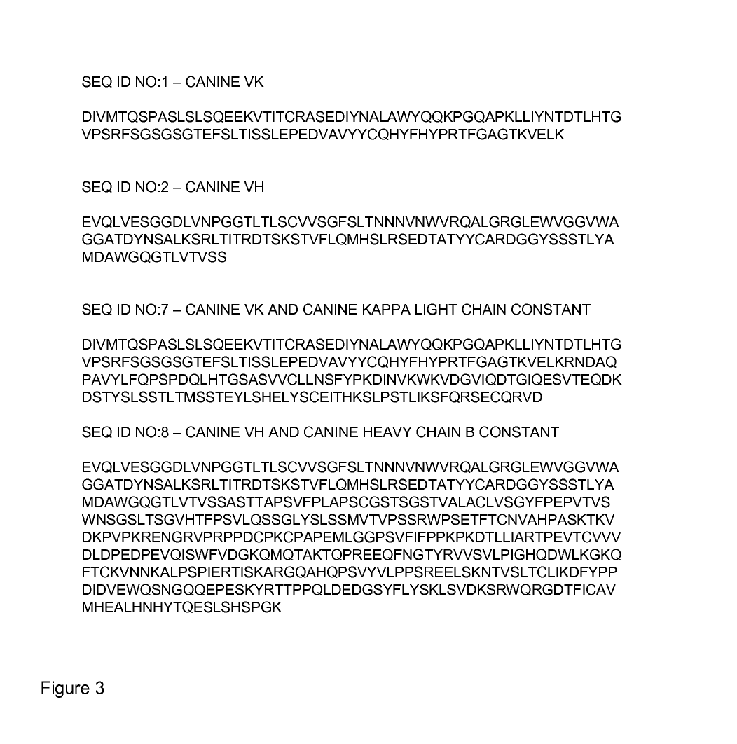

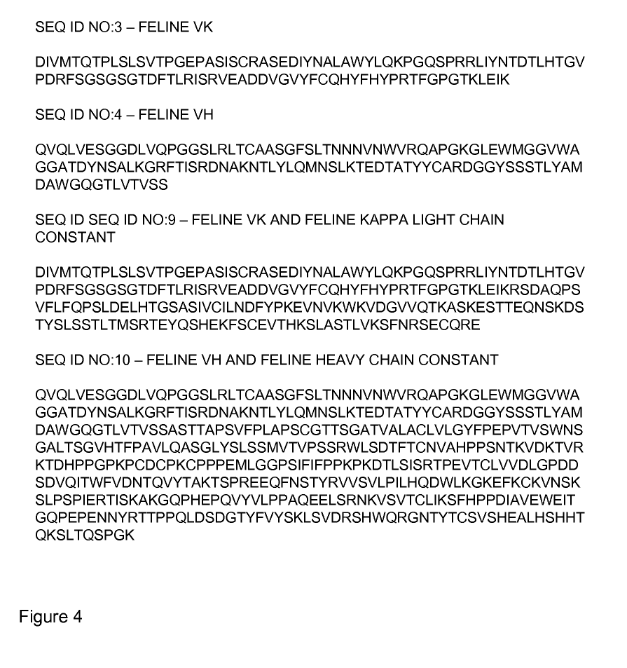

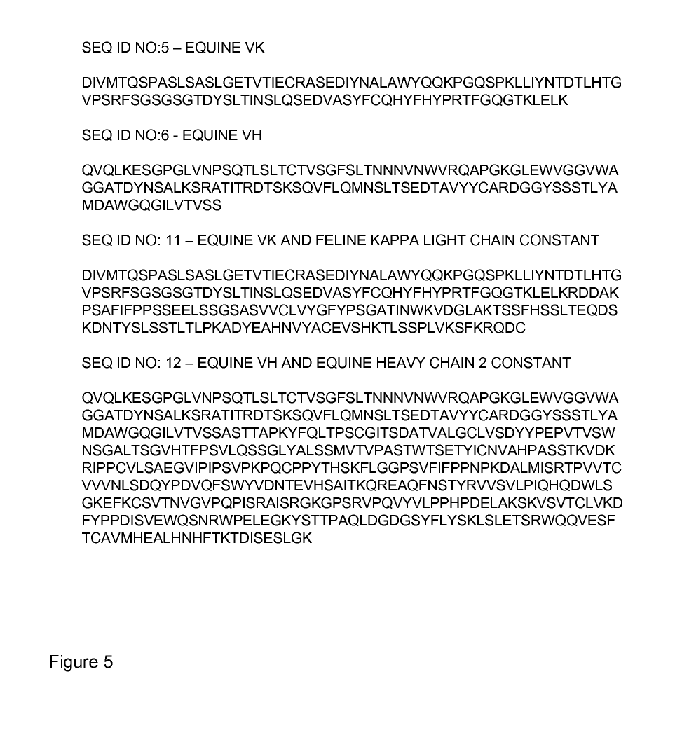

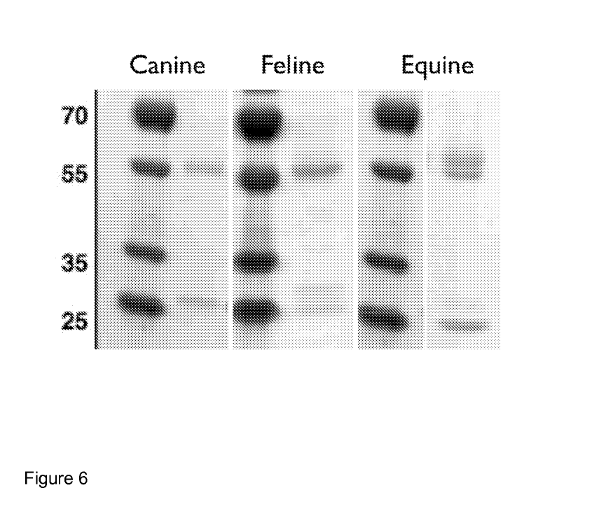

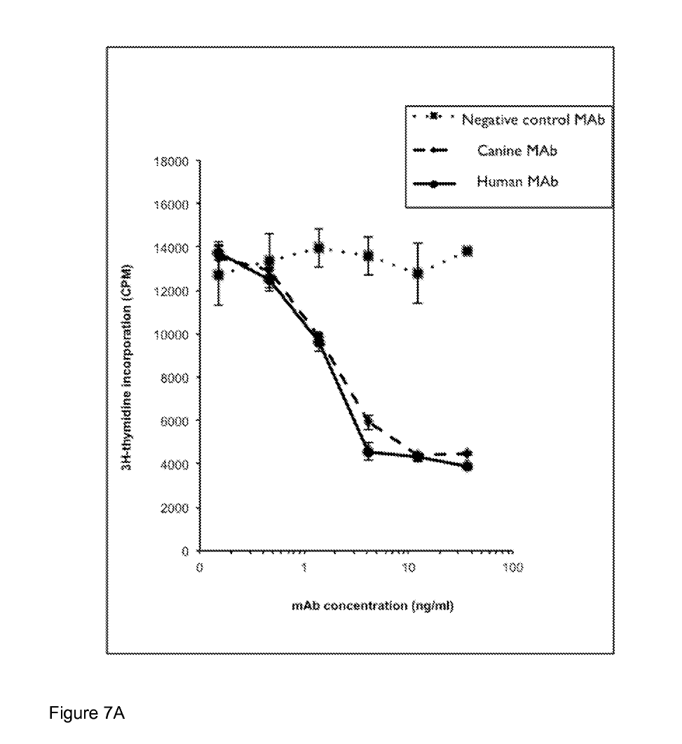

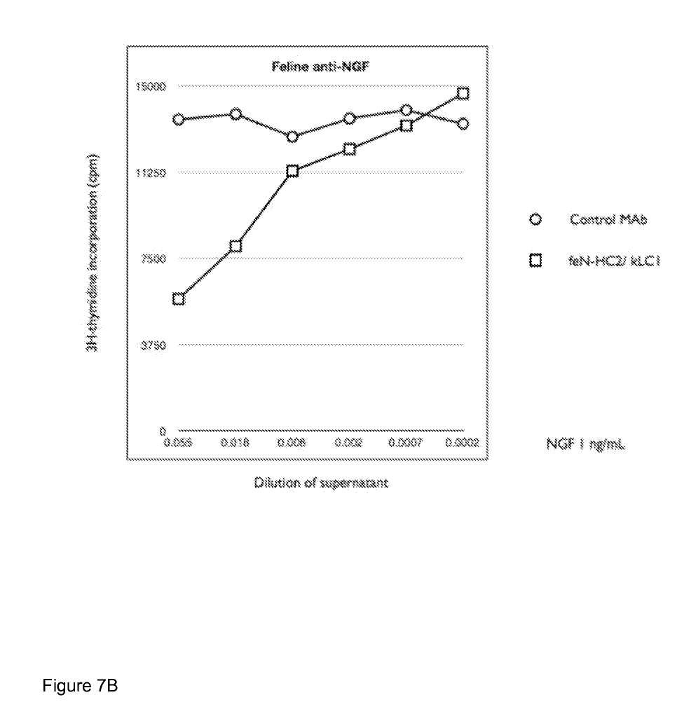

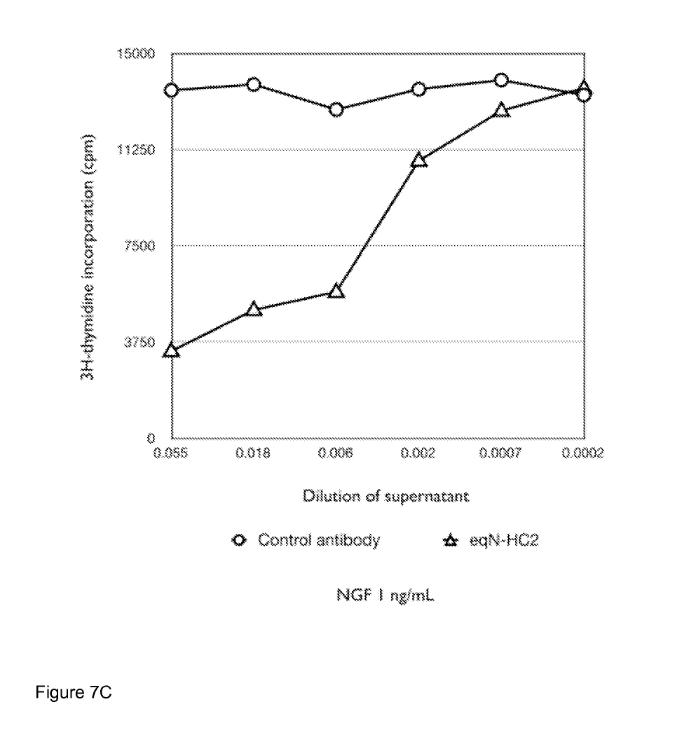

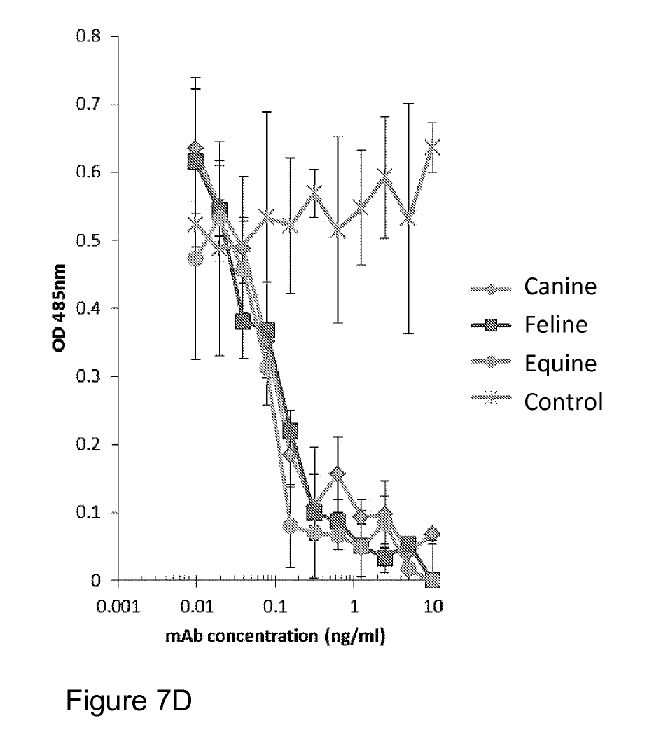

The variable domains for the canine, feline and equine versions of αD11 were expressed as full antibodies by C-terminal fusion to constant domains of each species as shown in FIGS. 3, 4 and 5 (variable domains SEQ ID NO 1-6, full antibodies SEQ ID NO 7-12) and co-expression of appropriate heavy and light chain pairs in expression vectors transfected into CHO cells. The supernatants containing the expressed heavy and light chain pairs as whole IgG were analyzed by SDS-PAGE and tested for their ability to inhibit proliferation of TF-1 cells by nerve growth factor in culture. Heavy and light chains were observed in Coomassie Blue stained SDS-PAGE gels (FIG. 6) and the supernatants were able to inhibit the proliferation of TF-1 cells induced by NGF (FIG. 7). By way of comparison, a purified sample of the caninised variant of αD11 was as effective at inhibiting NGF activity as a humanised version (Pavone et al. WO 2006/131951) of αD11 (FIG. 5a) illustrating that the PETising technique described herein does not lead to a reduction in the efficacy of the antibody structure per se, by comparison with the standard method of CDR grafting used to humanize the antibody by Pavone et al. The similar bioactivity of canine, feline and equine versions of αD11 is further illustrated in FIG. 7D.

TABLE 1
Light chain variable domain FR1 residues

| | Kabat | | | | |
|---|---|---|---|---|---|
| Light | light | | | | |
| chain | chain | | | | |
| FR1 | numbering | Canine | Feline | Equine | |
| position | position | VK | VK | VK | Human VK |
| 1 | 1 | DEA | DEN | DEGKV | DEAKY |
| 2 | 2 | ILV | VIPT | VIFNST | IVK |
| 3 | 3 | V | VEM | VMAGI | VQREAL |
| 4 | 4 | ML | MLI | MLVQ | MLC |
| 5 | 5 | MTI | T | TAI | TI |
| 6 | 6 | QE | Q | Q | Q |
| 7 | 7 | TSA | TS | STF | ST |
| 8 | 8 | P | P | P | PA |
| 9 | 9 | LAPRS | L | EDAPS | SGLADFPT |
| 10 | 10 | SP | SF | STFL | STFAL |
| 11 | 11 | L | L | LSV | LVSP |
| 12 | 12 | SA | SPA | ATESV | SAPH |
| 13 | 13 | VLA | V | VALQT | AVLR |
| 14 | 14 | STR | TIA | SAPT | ST |
| 15 | 15 | PQR | P | LPIR | VPLQR |
| 16 | 16 | EDGR | G | GR | GL |
| 17 | 17 | E | ED | QE | DEQGN |
| 18 | 18 | PTLKEAS | PSA | RSGKT | RPGTAS |
| 19 | 19 | AV | AV | VA | VASI |
| 20 | 20 | STF | S | ETDV | T |
| 21 | 21 | I | IF | MIVLT | IL |
| 22 | 22 | STY | SF | KSRETNQL | TSN |
| 23 | 23 | CY | C | C | C |

TABLE 2
Light chain variable domain FR2 residues

| | Kabat | | | | |
|---|---|---|---|---|---|
| Light | light | | | | |
| Chain | chain | | | | |
| FR2 | numbering | Canine | Feline | Equine | Human |
| Position | system | VK | VK | VK | VK |
| 1 | 35 | W | W | W | W |
| 2 | 36 | FYILS | YF | YFH | YFL |
| 3 | 37 | RQLIM | LFR | QRS | QHLR |
| 4 | 38 | QH | Q | QHKVCRS | QHEVGR |
| | | | | Y | |
| 5 | 39 | KR | K | KRV | KRISQT |
| 6 | 40 | PSA | P | PSAIL | PALSQ |
| 7 | 41 | GD | G | G | GE |
| 8 | 42 | QH | QR | QE | KQNRHT |
| 9 | 43 | SATP | S | ARSTVP | APSTGV |
| 10 | 44 | P | P | PL | P |
| 11 | 45 | QKER | R | KERIL | KRNQES |
| 12 | 46 | RLPGASD | RL | RQLAHG | LFRSVM |
| | | TI | | WEK | |
| 13 | 47 | LR | L | LVIFM | LV |
| 14 | 48 | ILT | IM | IFMVT | ILVMF |
| 15 | 49 | YFNSEVH | YHA | YSVFDEQ | YFSH |
| | | | | RTWACG | |
| | | | | HL | |

TABLE 3
Light chain variable domain FR3 residues

| | Kabat | | | | |
|---|---|---|---|---|---|
| Light | light | | | | |
| chain | chain | | | | |
| FR3 | numbering | Canine | Feline | Equine | Human |
| position | position | VK | VK | VK | VK |
| 1 | 57 | GA | GR | GDF | G |
| 2 | 58 | VA | V | VFA | VI |
| 3 | 59 | PS | P | PLS | PST |
| 4 | 60 | DSE | D | DAESGL | SDALEP |
| | | | | | W |
| 5 | 61 | RG | R | R | RM |
| 6 | 62 | FLVI | FI | FLY | FI |
| 7 | 63 | SIA | ST | SCFGNRT | SGTV |
| 8 | 64 | GA | G | GA | GAP |
| 9 | 65 | SR | S | SGRDEKT | SGI |
| | | | | W | |
| 10 | 66 | G | G | GRAV | GERV |
| 11 | 67 | S | S | SFYTA | SAP |
| 12 | 68 | GV | G | GET | GAS |
| 13 | 69 | TA | TAS | TASW | TAP |
| 14 | 70 | DE | D | DE | DEVS |
| 15 | 71 | FC | F | FY | FYH |
| 16 | 72 | TSR | TIA | TSAVY | TSIAN |
| 17 | 73 | LF | L | LFIP | LFHS |
| 18 | 74 | RTKE | RTK | TISAV | TKIAE |
| 19 | 75 | I | I | IV | IMSV |
| 20 | 76 | STG | SAGT | SNDTG | SNTAD |
| 21 | 77 | RGST | RG | SPTEDNR | SRGDNIP |
| | | | | | T |
| 22 | 78 | VLA | VM | LVFP | LVM |
| 23 | 79 | EVG | EQ | QER | QEKHLR |
| 24 | 80 | APD | AVPT | AEST | PAS |
| 25 | 81 | DEGINA | DE | ETAGD | ED |
| 26 | 82 | DG | D | DN | DN |
| 27 | 82A | AVTGS | VIHL | VAEGLS | FVSI |
| 28 | 82B | GA | G | AG | AG |
| 29 | 82C | VIL | V | IVTDSMN | TVSI |
| | | | | LEFGY | |
| 30 | 83 | Y | Y | YC | Y |
| 31 | 84 | YHFC | YF | YFHSTV | YF |
| | | | | W | |
| 32 | 85 | C | C | C | C |

TABLE 4
Light chain variable domain FR4 residues

| Light | Kabat | | | | |
|---|---|---|---|---|---|
| chain | light | | | | |
| FR4 | chain FR4 | Canine | Feline | Equine | Human |
| position | position | VK | VK | VK | VK |
| 1 | 95 | FLS | FS | FIL | FL |
| 2 | 96 | GS | G | G | GA |
| 3 | 97 | AQPTK | QP | QAL | QGP |
| 4 | 98 | GE | G | G | G |
| 5 | 99 | TPA | T | TS | TA |
| 6 | 100 | KQSNH | KHQEST | KNRM | KRT |
| 7 | 101 | VLW | L | LVM | VLI |
| 8 | 102 | DERVG | ED | EADK | EDP |
| 9 | 103 | LIM | IVML | ILVFM | ITVFM |
| 10 | 104 | KR | KRDT | KERTAGI | KRTEN |
| | | | | QV | |

TABLE 5
Heavy chain variable domain FR1 residues

| | Kabat | | | | |
|---|---|---|---|---|---|
| Heavy | heavy | | | | |
| chain | chain | | | | |
| FR1 | numbering | Canine | Feline | Equine | Human |
| position | position | VH | VH | VH | VH |
| 1 | 1 | EDG | QDEH | Q | QEHL |
| 2 | 2 | VGLEIM | VE | V | VLGIM |
| 3 | 3 | QHRAVEK | LQR | Q | QRHIKY |
| | | LPS | | | |
| 4 | 4 | LVP | LV | L | LV |
| 5 | 5 | VALEM | VM | KQ | VQLEK |
| 6 | 6 | EQA | QED | E | EQDV |
| 7 | 7 | SFLT | S | S | SWP |
| 8 | 8 | GA | G | G | GAE |
| 9 | 9 | GEA | GAR | P | GAPT |
| 10 | 10 | DAGNET | EDN | GD | GEDARV |
| | | W | | | |
| 11 | 11 | LQRVW | LVR | LQ | LVFPSW |
| 12 | 12 | VAIMK | VRSK | VM | VKLIMRA |
| | | | | | GT |
| 13 | 13 | KRNQ | KTQENR | KMNR | KQERNT |
| 14 | 14 | PFT | PT | PIS | PALR |
| 15 | 15 | GAETS | GE | SAG | GSER |
| 16 | 16 | GEA | GATE | QE | GESRAQT |
| | | | | | VD |
| 17 | 17 | STP | SA | TA | STA |
| 18 | 18 | LRV | LV | L | LVRE |
| 19 | 19 | RKTGV | RKES | ST | RKSTHI |
| 20 | 20 | LIV | ILP | L | LVIR |
| 21 | 21 | SY | FTS | TVIS | STFN |
| 22 | 22 | C | C | C | C |
| 23 | 23 | VLAIEK | VKAMQ | TASF | AKTEVSIR |
| | | | | | QDG |
| 24 | 24 | ATVGIS | ATDV | VI | AVGITS |
| 25 | 25 | SPGT | S | ST | SYCF |
| 26 | 26 | GDRT | GA | GA | GVAR |
| 27 | 27 | FLIDSTV | FYL | LFAGIMN | GDAVY |
| | | | | QS | |

TABLE 6
Heavy chain variable domain FR2 residues

| | Kabat | | | | |
|---|---|---|---|---|---|
| Heavy | heavy | | | | |
| chain | chain | | | | |
| FR2 | numbering | Canine | Feline | Equine | Human |
| position | system | VH | VH | VH | VH |
| 1 | 36 | WC | W | W | W |
| 2 | 37 | VIAFL | VLWFIA | VL | VILFA |
| 3 | 38 | RQ | RCH | R | RFP |
| 4 | 39 | QLHRE | Q | Q | QHRL |
| 5 | 40 | ASTGPV | APVTS | APSV | APSVML |
| | | DC | | | HDGT |
| 6 | 41 | PL | P | P | PSAR |
| 7 | 42 | GERL | GAES | G | GSEQRW |
| 8 | 43 | KREGA | KQTE | KRW | KQRNT |
| | | MQ | | | |
| 9 | 44 | GERDT | G | GR | GRK |
| | | V | | | |
| 10 | 45 | LTPFM | LFP | LPW | LHIP |
| 11 | 46 | QEHDL | QE | E | EQVADK |
| | | PRK | | | |
| 12 | 47 | WLCSY | WCL | FYWEHR | W |
| | | FM | | SV | |
| 13 | 48 | VLIFM | VMI | VI | VMIL |
| 14 | 49 | ATSGLV | AGTS | GASD | GSA |

TABLE 7
Heavy chain variable domain FR3 residues

| | Kabat | | | | |
|---|---|---|---|---|---|
| Heavy | heavy | | | | |
| chain | chain | | | | |
| FR3 | numbering | Canine | Feline | Equine | Human |
| position | position | VH | VH | VH | VH |
| 1 | 66 | RQ | RQK | R | RQHG |
| 2 | 67 | FVL | FL | AVGCIT | FVILMAT |
| 3 | 68 | TAIS | TIA | SRIDMNT | TISAVH |
| 4 | 69 | IVLMT | ILVM | IV | IMVFALT |
| 5 | 70 | SAFT | ST | TSIL | STLNV |
| 6 | 71 | RKA | RAITKG | KRES | RAVLTISP |
| | | | V | | EKW |
| 7 | 72 | DEN | D | DEN | DN |
| 8 | 73 | NDTSIGL | TNDAS | TSAEIPY | NTDEKA |
| | | | | | MIS |
| 9 | 74 | AGVSDP | ASDTG | SEGKT | SAVYGT |
| | | T | | | |
| 10 | 75 | KRENQG | KTRGQ | KELNQR | KTRIEQA |
| | | MT | E | | NS |
| 11 | 76 | NDSKHR | NDK | SNGKR | NSKTDAG |
| 12 | 77 | TMIAS | TIA | QERH | TQSIHEF |
| | | | | | R |
| 13 | 78 | LVMAIQ | LAVG | VILSAF | LAFVMSI |
| | | F | | | T |
| 14 | 79 | YFSHT | YFASV | YLSTVFR | YSFHDTV |
| | | | WD | | W |
| 15 | 80 | LIM | LM | LV | LM |
| 16 | 81 | QHEDRA | QELRD | TIQA | QEKNRH |
| | | | HV | | DST |
| 17 | 82 | ML | MLT | LMV | MLWIV |
| 18 | 82A | NDSTHK | NSDTG | NTDKRS | NSTRHD |
| | | PRE | RH | | |
| 19 | 82B | SGDRNT | SNIRT | STADEGK | SRNDGTA |
| | | | | M | IL |
| 20 | 82C | LV | L | LVM | LV |
| 21 | 83 | RTGKSI | KRTGQ | TS | RTKQ |
| 22 | 84 | AVDTSG | STPAIV | SGDER | ASITPVG |
| | | P | | | DLN |
| 23 | 85 | EDAV | EATDGS | EDG | EADSGK |
| 24 | 86 | D | D | D | DN |
| 25 | 87 | TASM | T | TA | TSA |
| 26 | 88 | AGV | AGS | AS | AGS |
| 27 | 89 | VMILFT | TVMIA | VD | VIMLT |
| | | KQY | | | |
| 28 | 90 | YH | YH | Y | Y |
| 29 | 91 | YFHAGT | YHFC | YFWAI | YFH |
| 30 | 92 | C | CR | C | C |
| 31 | 93 | AVTGMR | AITSVG | ATVGEIS | AVTGSLM |
| | | SCLPK | M | | |
| 32 | 94 | KRSNGA | RKSTIV | GRAEHIK | RKGTSNV |
| | | TPDQVEI | PNG | S | AHIY |
| | | M | | | |

TABLE 8
Heavy chain variable domain FR4 residues

| | Kabat | | | | |
|---|---|---|---|---|---|
| Heavy | heavy | | | | |
| chain | chain | | | | |
| FR4 | numbering | Canine | Feline | Equine | Human |
| position | position | VH | VH | VH | VH |
| 1 | 103 | WL | WRCL | W | WCISY |
| 2 | 104 | GAS | GRA | G | GLS |
| 3 | 105 | QPHRD | QPHVR | QP | QKRHLAE |
| | | | | | P |
| 4 | 106 | G | GD | G | GPR |
| 5 | 107 | TASIN | ATVIS | I | TISAEP |
| 6 | 108 | LSQPR | LIQMST | L | LTMVPQA |
| | | | | | CN |
| 7 | 109 | VLIP | VI | V | VICFGLW |
| 8 | 110 | TFIASLP | TAIR | T | TISADLV |
| | | Y | | | |
| 9 | 111 | VA | VIG | V | VGILP |
| 10 | 112 | SACPT | STP | S | SFTW |
| 11 | 113 | SLAP | SQPA | — | SPFGT |

### Example 2

### Production of Anti-Human NGF De-Immunised Antibody

The PETising technique can also be used to convert antibodies for human use (termed hereinbefore as “re-humanising”) having alternative human amino acid heavy and light chain sequences to those of Pavone et al. using the method described above and the pool of human derived framework region position specific amino acid residues shown in Tables 1-8.

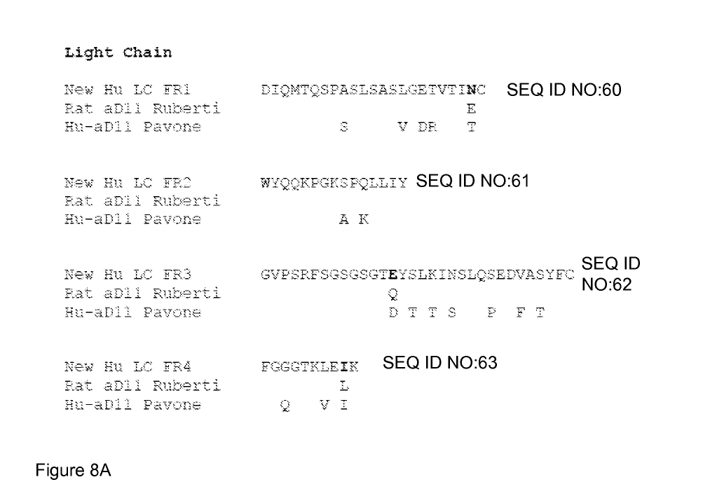

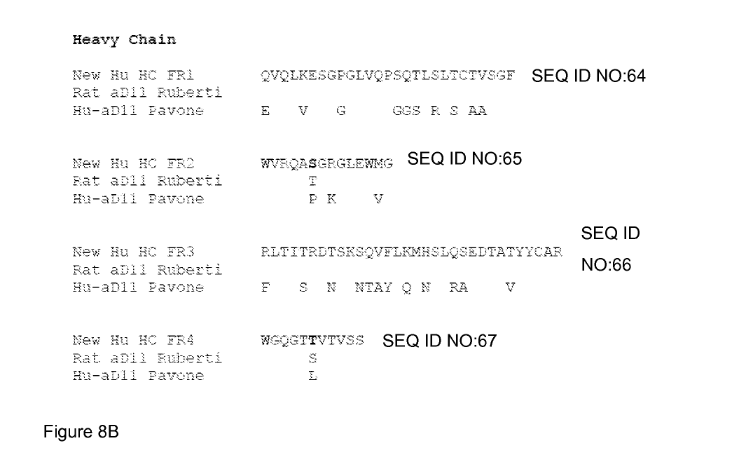

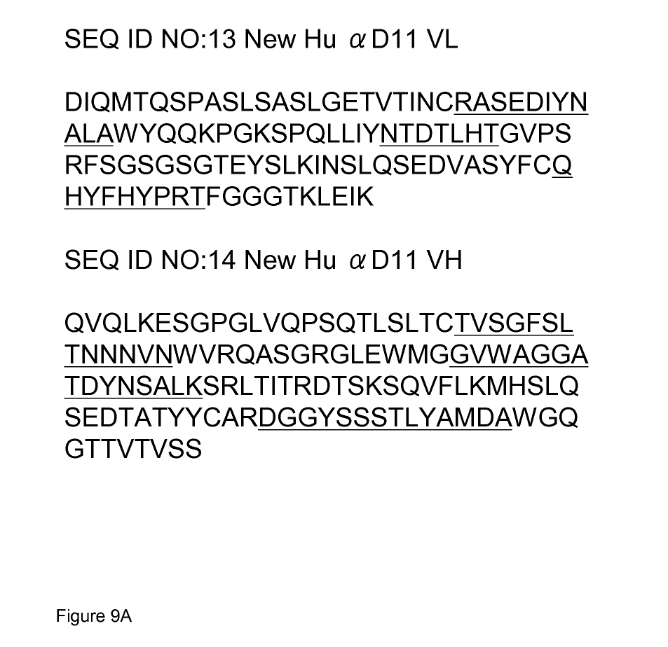

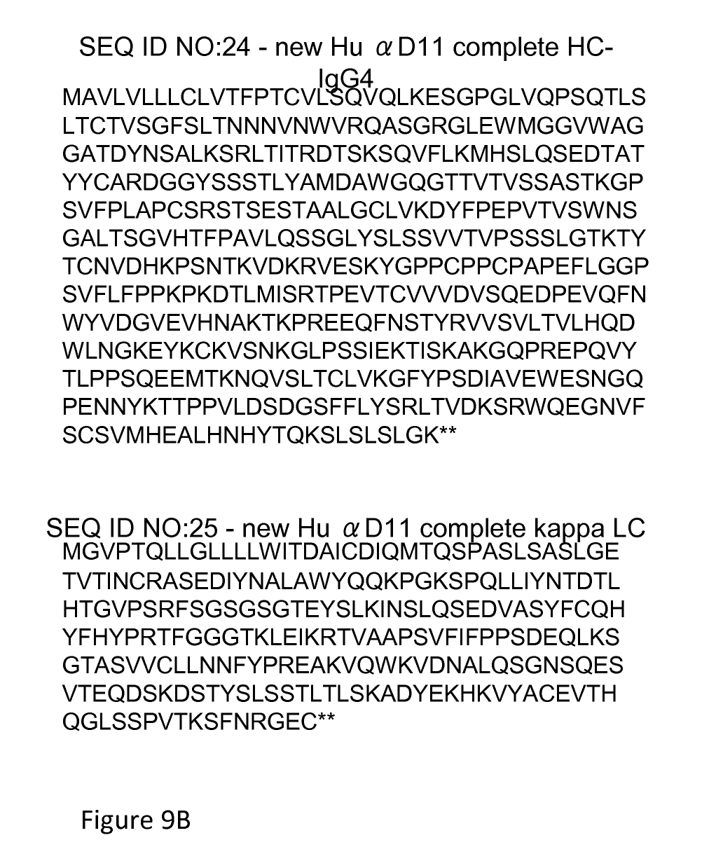

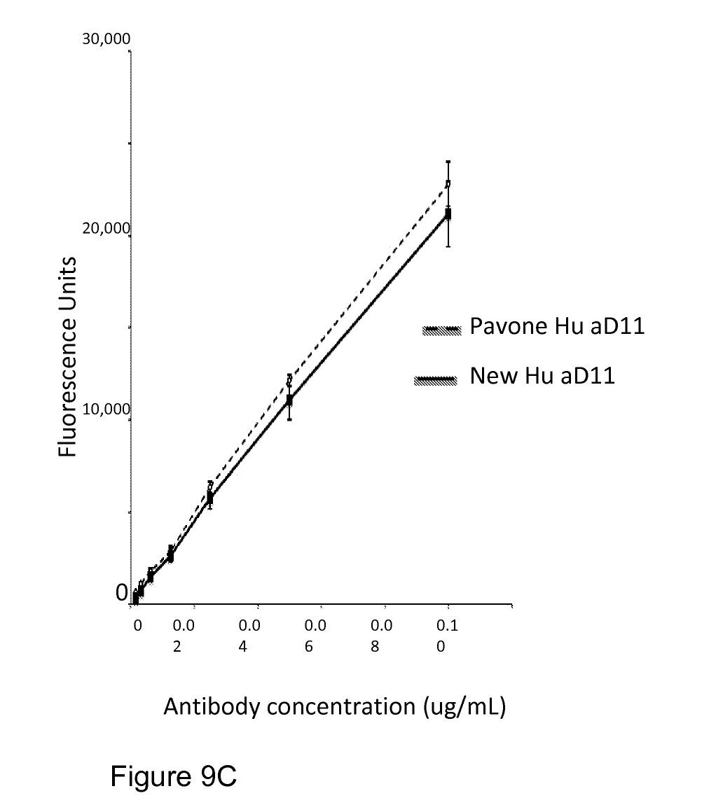

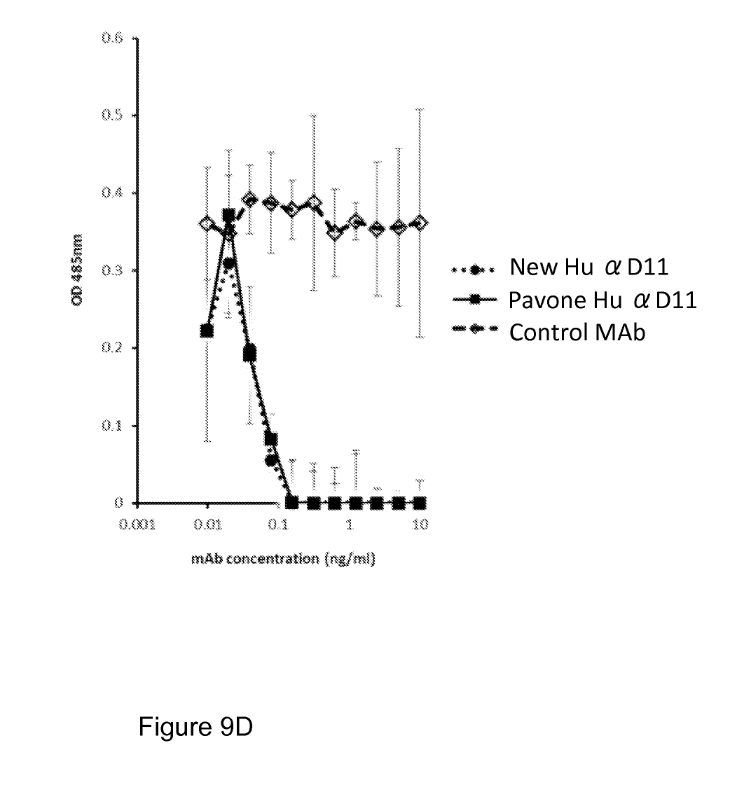

A comparison of the re-humanised framework sequences of the light chain (FIG. 8A) and heavy chain (FIG. 8B) are shown in addition to the resultant VH and VL sequences (FIG. 9A). It is apparent that far fewer changes need be made to humanize αD11 MAb using the method disclosed herein (3 amino acid changes to the framework regions) than those used in the standard CDR grafting method by Pavone and colleagues (43 amino acid changes).

The protein sequences of the variable heavy chain (VH) and light chain (VL) variants of re-humanised αD11 antibodies (New-Hu αD11, FIG. 9A) were designed with N-terminal signal sequences from rat αD11 and C-terminal human IgG constant domains from IgG4 heavy chain and human kappa light chain respectively (FIG. 9B). Synthetic genes encoding these were prepared by oligonucleotide-based gene synthesis and each was subcloned into the mammalian expression vector pcDNA3.1+. Co-transfection into CHO cells and purification from cell supernatants using Protein A chromatography yielded purified antibodies. The antibodies were tested for binding to NGF by ELISA and compared to binding by the humanised variant of αD11 described by Pavone et al. (WO 06/131951, designed by CDR grafting). As can be seen from the ELISA results shown in FIG. 9C, the New Hu αD11 variant of the present patent has binding to NGF that is indistinguishable to that described by Pavone et al. The antibodies described in FIG. 9C were tested for inhibition of NGF by the method described in FIG. 7C. Both humanised antibodies showed equivalent bioactivity.

### Example 3

### Production of Canine Antibodies Having Binding Specificity to Canine TNF

By way of further example, the PETising method of this patent was used with a human antibody D2E7 that binds tumour necrosis factor as the starting point in order to make a canine variant thereof.

Tables 9-16, shown below, illustrate the alignment of the framework regions of the light and heavy chain variable domains of the human monoclonal antibody D2E7 with the pool of the amino acid residues defined for each position of the canine immunoglobulin framework sequences which (as shown in Tables 1-8). Certain residues (marked * in Table 9) previously thought to be foreign at that Kabat sequence position are now considered natural to canines, hence the changes shown at the positions marked * would now no longer be made, or an alternative, more conserved, residue may be chosen (marked ** in Table 10). Based on this additional information, three light chain positions would be unchanged, including Ser9, Ala13 and Gly16 (marked * in Table 9). Light chain His42 would be an alternative choice of residue at that position (marked ** in Table 10). Modifications of the caninised D2E7 framework residues at these positions, and antibodies comprising such modifications, are intended to be within the scope of this invention. Such antibodies would comprise a light chain variable domain having the sequence shown in SEQ ID NO:15 with the exception that a serine residue is provided at position 9, an alanine residue is provided at position 13 and a glycine residue is provided at position 16 in place of the residues shown at these positions in SEQ ID NO:15. Optionally a histidine residue may be provided at position 42 of SEQ ID NO:15 in place of glutamine. A modified sequence showing these changes is provided as SEQ ID NO:71. The invention extends to antibodies comprising a light chain variable domain comprising SEQ ID NO:71, and antigen binding fragments derived from same.

TABLE 9
Caninised light chain FR1 sequence of human
derived D2E7 monoclonal antibody

| | Kabat | Human | Canine VK | Canine |
|---|---|---|---|---|
| Light chain | light chain | MAb | amino acid | PETised |
| FR1 | numbering | D2E7 | pool | D2E7 |
| position | position | VL | sequences | VL |
| 1 | 1 | D | DEA | D |
| 2 | 2 | I | ILV | I |
| 3 | 3 | Q | V | V |
| 4 | 4 | M | ML | M |
| 5 | 5 | T | MTI | T |
| 6 | 6 | Q | QE | Q |
| 7 | 7 | S | TSA | S |
| 8 | 8 | P | P | P |
| 9 | 9 | S | LAPRS | A* |
| 10 | 10 | S | SP | S |
| 11 | 11 | L | L | L |
| 12 | 12 | S | SA | S |
| 13 | 13 | A | VLA | L * |
| 14 | 14 | S | STR | S |
| 15 | 15 | V | PQR | Q |
| 16 | 16 | G | EDGR | E * |
| 17 | 17 | D | E | E |
| 18 | 18 | R | PTLKEAS | K |
| 19 | 19 | V | AV | V |
| 20 | 20 | T | STF | T |
| 21 | 21 | I | I | I |
| 22 | 22 | T | STY | T |
| 23 | 23 | C | CY | C |

TABLE 10
Caninised light chain FR2 sequence of human
derived D2E7 monoclonal antibody

| | Kabat light | Human | Canine VK | Canine |
|---|---|---|---|---|
| Light Chain | chain | MAb | amino acid | PETised |
| FR2 | numbering | D2E7 | pool | D2E7 |
| Position | system | VL | sequences | VL |
| 1 | 35 | W | W | W |
| 2 | 36 | Y | FYILS | Y |
| 3 | 37 | Q | RQLIM | Q |
| 4 | 38 | Q | QH | Q |
| 5 | 39 | K | KR | K |
| 6 | 40 | P | PSA | P |
| 7 | 41 | G | GD | G |
| 8 | 42 | K | QH** | Q |
| 9 | 43 | A | SATP | A |
| 10 | 44 | P | P | P |
| 11 | 45 | K | QKER | K |
| 12 | 46 | L | RLPGASDTI | L |
| 13 | 47 | L | LR | L |
| 14 | 48 | I | ILT | I |
| 15 | 49 | Y | YFNSEVHW | Y |

TABLE 11
Caninised light chain FR3 sequence of
human derived D2E7 monoclonal antibody

| | Kabat | | Canine VK | |
|---|---|---|---|---|
| Light | light | Human | amino | Canine |
| chain | chain | MAb | acid | PETised |
| FR3 | numbering | D2E7 | pool | D2E7 |
| position | position | VL | sequences | VL |
| 1 | 57 | G | GA | G |
| 2 | 58 | V | VA | V |
| 3 | 59 | P | PS | P |
| 4 | 60 | S | DSE | S |
| 5 | 61 | R | RG | R |
| 6 | 62 | F | FLVI | F |
| 7 | 63 | S | SIA | S |
| 8 | 64 | G | GA | G |
| 9 | 65 | S | SR | S |
| 10 | 66 | G | G | G |
| 11 | 67 | S | S | S |
| 12 | 68 | G | GV | G |
| 13 | 69 | T | TA | T |
| 14 | 70 | D | DE | D |
| 15 | 71 | F | FC | F |
| 16 | 72 | T | TSR | T |
| 17 | 73 | L | LF | L |
| 18 | 74 | T | RTKE | T |
| 19 | 75 | I | I | I |
| 20 | 76 | S | STG | S |
| 21 | 77 | S | RGST | S |
| 22 | 78 | L | VLA | L |
| 23 | 79 | Q | EVG | E |
| 24 | 80 | P | APD | P |
| 25 | 81 | E | DEGINA | E |
| 26 | 82 | D | DG | D |
| 27 | 82A | V | AVTGS | V |
| 28 | 82B | A | GA | A |
| 29 | 82C | T | VIL | V |
| 30 | 83 | Y | Y | Y |
| 31 | 84 | Y | YHFC | Y |
| 32 | 85 | C | C | C |

TABLE 12
Caninised light chain FR4 sequence of human
derived D2E7 monoclonal antibody

| | | Human | | Canine |
|---|---|---|---|---|
| Light chain | Kabat light | MAb | Canine VK | PETised |
| FR4 | chain FR4 | D2E7 | amino acid | D2E7 |
| position | position | VL | pool sequences | VL |
| 1 | 95 | F | FLS | F |
| 2 | 96 | G | GS | G |
| 3 | 97 | Q | AQPTK | Q |
| 4 | 98 | G | GE | G |
| 5 | 99 | T | TPA | T |
| 6 | 100 | K | KQSNH | K |
| 7 | 101 | V | VLW | V |
| 8 | 102 | E | DERVG | E |
| 9 | 103 | I | LIM | I |
| 10 | 104 | K | KR | K |

TABLE 13
Caninised heavy chain FR1 sequence of human
derived D2E7 monoclonal antibody

| | Kabat heavy | Human | | Canine |
|---|---|---|---|---|
| Heavy chain | chain | MAb | Canine VH | PETised |
| FR1 | numbering | D2E7 | amino acid | D2E7 |
| position | position | VH | pool sequences | VH |
| 1 | 1 | E | EDG | E |
| 2 | 2 | V | VGLEIM | V |
| 3 | 3 | Q | QHRAVEKLPS | Q |
| 4 | 4 | L | LVP | L |
| 5 | 5 | V | VALEM | V |
| 6 | 6 | E | EQA | E |
| 7 | 7 | S | SFLT | S |
| 8 | 8 | G | GA | G |
| 9 | 9 | G | GEA | G |
| 10 | 10 | G | DAGNETW | G |
| 11 | 11 | L | LQRVW | L |
| 12 | 12 | V | VAIMK | V |
| 13 | 13 | Q | KRNQ | Q |
| 14 | 14 | P | PFT | P |
| 15 | 15 | G | GAETS | G |
| 16 | 16 | R | GEA | G |
| 17 | 17 | S | STP | S |
| 18 | 18 | L | LRV | L |
| 19 | 19 | R | RKTGV | R |
| 20 | 20 | L | LIV | L |
| 21 | 21 | S | SY | S |
| 22 | 22 | C | C | C |
| 23 | 23 | A | VLAIEK | A |
| 24 | 24 | A | ATVGIS | A |
| 25 | 25 | S | SPGT | S |
| 26 | 26 | G | GDRT | G |
| 27 | 27 | F | FLIDSTV | F |

TABLE 14
Caninised heavy chain FR2 sequence of human
derived D2E7 monoclonal antibody

| Heavy | Kabat heavy | Human | Canine VH | Canine |
|---|---|---|---|---|
| chain | chain | MAb | amino acid | PETised |
| FR2 | numbering | D2E7 | pool | D2E7 |
| position | system | VH | sequences | VH |
| 1 | 36 | W | WC | W |
| 2 | 37 | V | VIAFL | V |
| 3 | 38 | R | RQ | R |
| 4 | 39 | Q | QLHRE | Q |
| 5 | 40 | A | ASTGPVDC | A |
| 6 | 41 | P | PL | P |
| 7 | 42 | G | GERL | G |
| 8 | 43 | K | KREGAMQ | K |
| 9 | 44 | G | GERDTV | G |
| 10 | 45 | L | LTPFM | L |
| 11 | 46 | E | QEHDLPRK | E |
| 12 | 47 | W | WLCSYFM | W |
| 13 | 48 | V | VLIF M | V |
| 14 | 49 | S | ATSGLV | S |

TABLE 15
Caninised heavy chain FR3 sequence of human
derived D2E7 monoclonal antibody

| | Kabat heavy | Human | Canine VH | Canine |
|---|---|---|---|---|
| Heavy | chain | D2E7 MAb | amino acid | PETised |
| chain FR3 | numbering | VH | pool | D2E7 |
| position | position | sequence | sequences | VH |
| 1 | 66 | R | RQ | R |
| 2 | 67 | F | FVL | F |
| 3 | 68 | T | TAIS | T |
| 4 | 69 | I | IVLMT | I |
| 5 | 70 | S | SAFT | S |
| 6 | 71 | R | RKA | R |
| 7 | 72 | D | DEN | D |
| 8 | 73 | N | NDTSIGL | N |
| 9 | 74 | A | AGVSDPT | A |
| 10 | 75 | K | KRENQGMT | K |
| 11 | 76 | N | NDSKHR | N |
| 12 | 77 | S | TMIAS | S |
| 13 | 78 | L | LVMAIQF | L |
| 14 | 79 | Y | YFSHT | Y |
| 15 | 80 | L | LIM | L |
| 16 | 81 | Q | QHEDRA | Q |
| 17 | 82 | M | ML | M |
| 18 | 82A | N | NDSTHKPR | N |
| | | | E | |
| 19 | 82B | S | SGDRNT | S |
| 20 | 82C | L | LV | L |
| 21 | 83 | R | RTGKSI | R |
| 22 | 84 | A | AVDTSGP | A |
| 23 | 85 | E | EDAV | E |
| 24 | 86 | D | D | D |
| 25 | 87 | T | TASM | T |
| 26 | 88 | A | AGV | A |
| 27 | 89 | V | VMILFTKQ | V |
| | | | Y | |
| 28 | 90 | Y | YH | Y |
| 29 | 91 | Y | YFHAGT | Y |
| 30 | 92 | C | C | C |
| 31 | 93 | A | AVTGMRSC | A |
| | | | LPK | |
| 32 | 94 | K | KRSNGATP | K |
| | | | DQVEIM | |

TABLE 16
Caninised heavy chain FR4 sequence of human
derived D2E7 monoclonal antibody

| | Kabat heavy | Human | Canine VH | Canine |
|---|---|---|---|---|
| Heavy | chain | D2E7 MAb | amino acid | PETised |
| chain FR4 | numbering | VH | pool | D2E7 |
| position | position | sequence | sequences | VH |
| 1 | 103 | W | WL | W |
| 2 | 104 | G | GAS | G |
| 3 | 105 | Q | QPHRD | Q |
| 4 | 106 | G | G | G |
| 5 | 107 | T | TASIN | T |
| 6 | 108 | L | LSQPR | L |
| 7 | 109 | V | VLIP | V |
| 8 | 110 | T | TFIASLPY | T |
| 9 | 111 | V | VA | V |
| 10 | 112 | S | SACPT | S |
| 11 | 113 | S | SLAP | S |

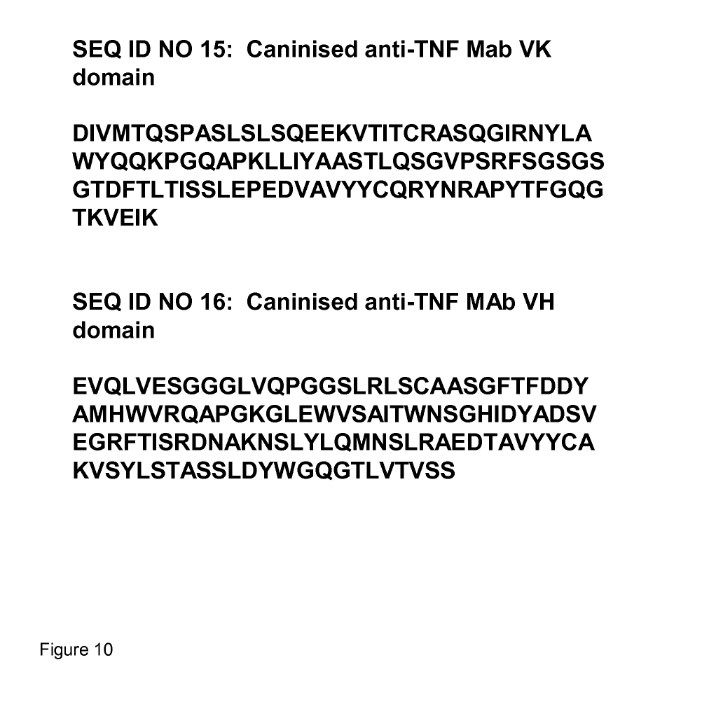

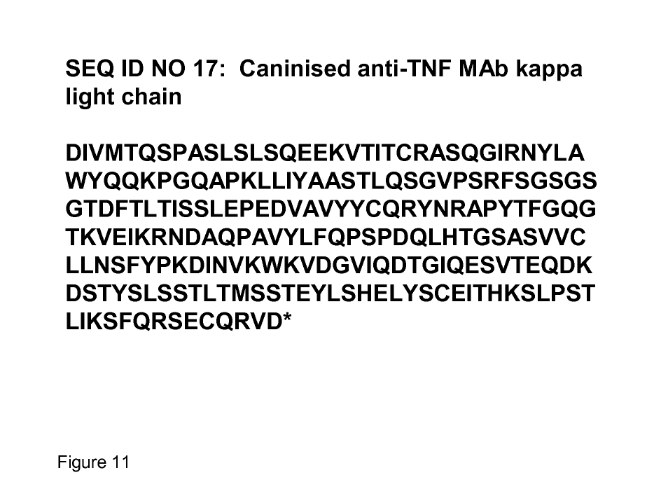

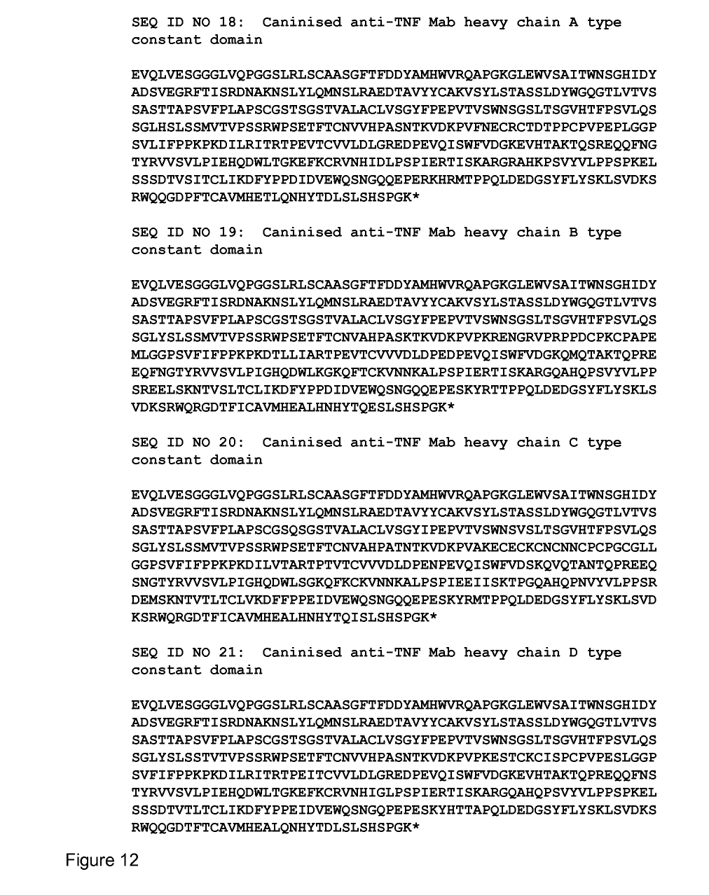

FIGS. 10, 11 and 12 illustrate variable domain (FIG. 10) and whole antibody (FIG. 11 (light chain) and FIG. 12 (heavy chain)) sequences encoding canine PETised Mab variants of D2E7 in SEQ ID NO:15 to SEQ ID NO:21. The sequences were built as DNAs using oligonucleotide synthesis and subcloned to expression vectors and transfected to CHO cells as above. In particular, the sequences of SEQ ID NO:17 (light chain) and SEQ ID NO:18-21 (heavy chain isotypes A, B, C and D) were designed and built as DNAs using oligonucleotide synthesis and subcloned to pcDNA3.1+ expression vectors and transfected in various combinations into CHO cells.

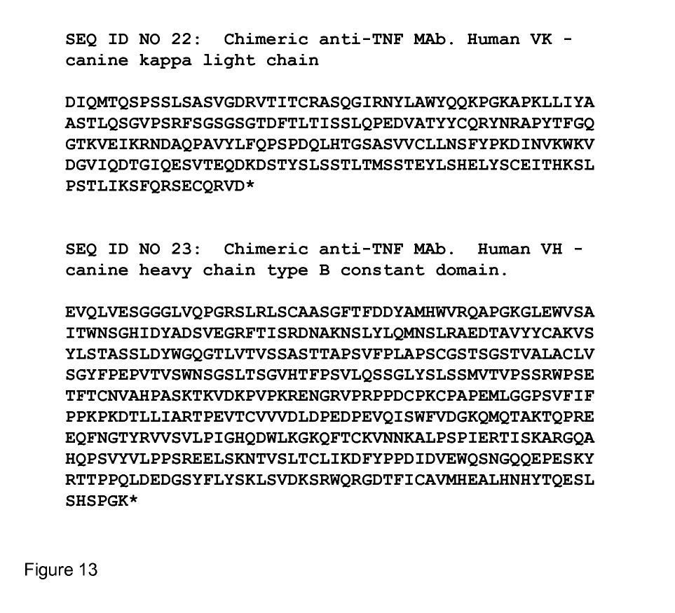

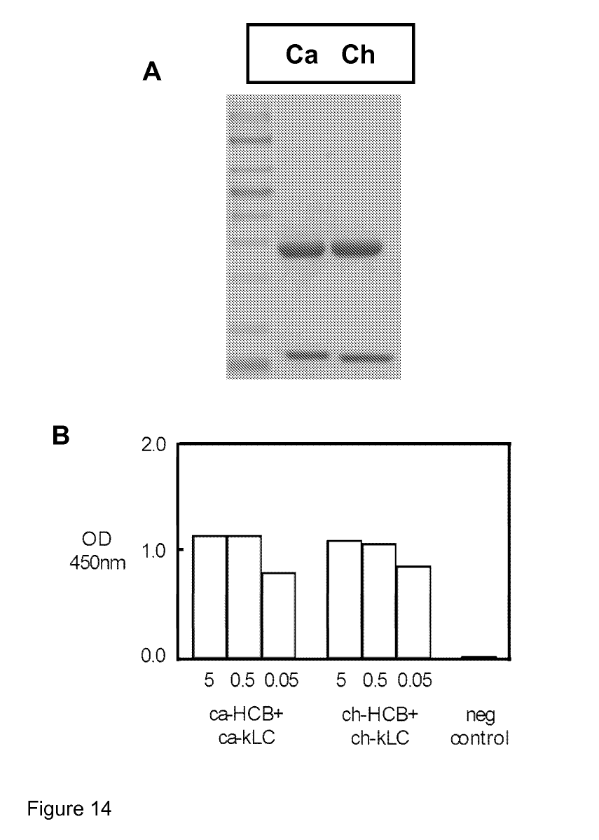

cDNAs encoding caninised anti-TNF monoclonal antibodies having the amino acid sequence of SEQ ID NO:17 (light chain) and SEQ ID NO:19 (heavy chain, isotype B) and a chimeric anti-TNF monoclonal antibody having a light chain with the amino acid of SEQ ID NO:22 and a heavy chain of SEQ ID NO:23 (FIG. 13) were subcloned into pcDNA3.1+ (Invitrogen/Life technologies) with amino-terminal secretory signal sequences (not shown). CHO cells were co-transfected with combinations of either caninised heavy and light chain sequences (ca-HCB+ca-kLC) or chimeric heavy and light chains (ch-HCB+ch-kLC). The resultant supernatants were purified on Protein A, analysed by SDS-PAGE (FIG. 14A) and tested for binding to canine TNF alpha (coated at 5 ug/ml; R&D systems) at the indicated concentrations of antibody (ug/ml) by ELISA and detected using anti-canine polyclonal antibody-horseradish peroxidase conjugate (Sigma A9042) (FIG. 14B). The negative control was the detection polyclonal antibody on coated antigen alone.

Purified antibodies were tested for their ability to inhibit canine TNF activity using 293-HEK cells transfected with pTRH1 to produce a TNF sensitive NF-kB-EGFP reporter cell line that responds to human TNF by fluorescence (Vince et al, Cell 131, 682, 2007). It was first demonstrated that canine TNF activates GFP expression in these cells (50% maximal stimulation at approximately 1 ng/ml) and then the canine antibodies shown in FIG. 14 were tested for their ability to inhibit 1 ng/mL canine TNF.

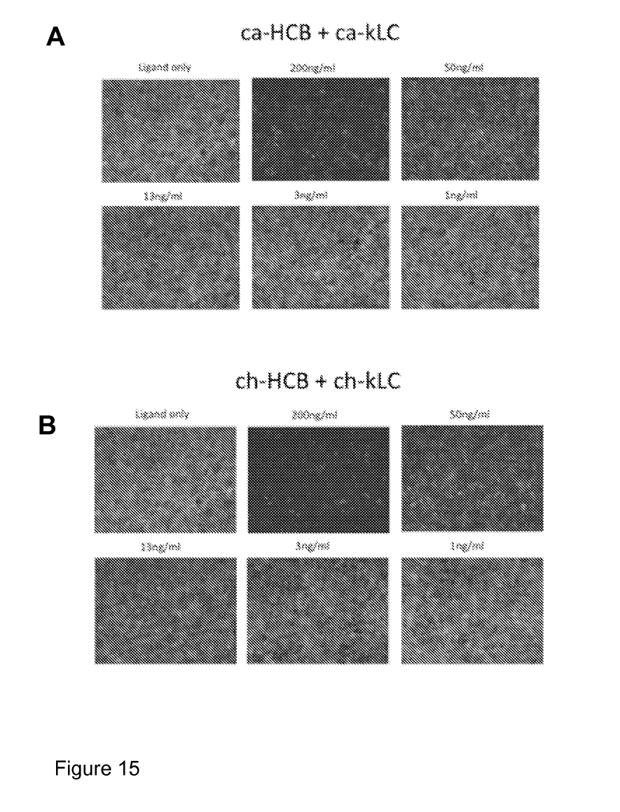

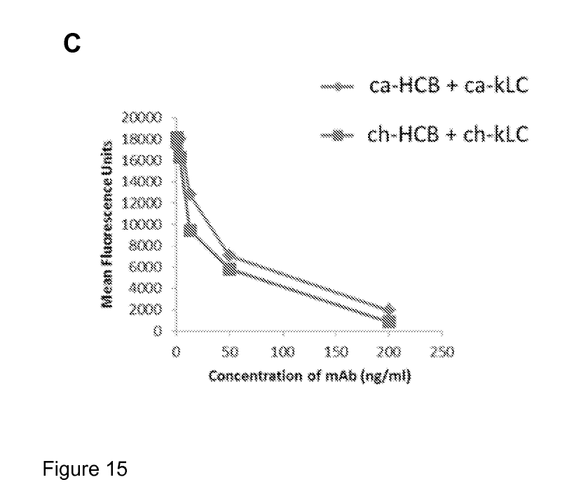

As shown in FIG. 15 (A-C), both the caninised and the chimeric antibodies were potent inhibitors of canine TNF in this assay.

Together these results showed that the caninised antibodies of the invention and the human-canine chimeric antibody bind canine TNF and are equipotent by both ELISA and inhibition assay, demonstrating that the caninisation process has produced a fully active canine version of the original D2E7 antibody.

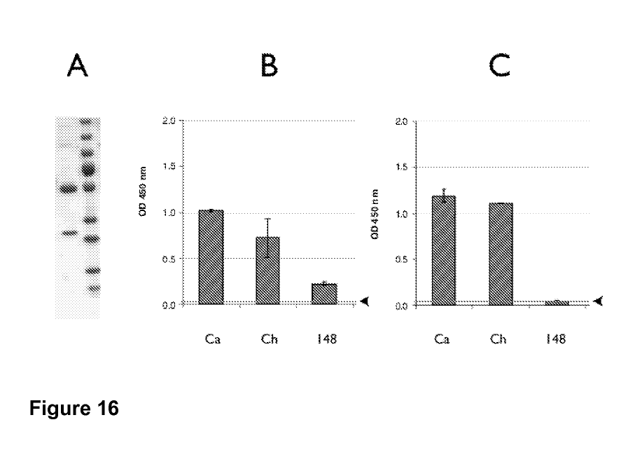

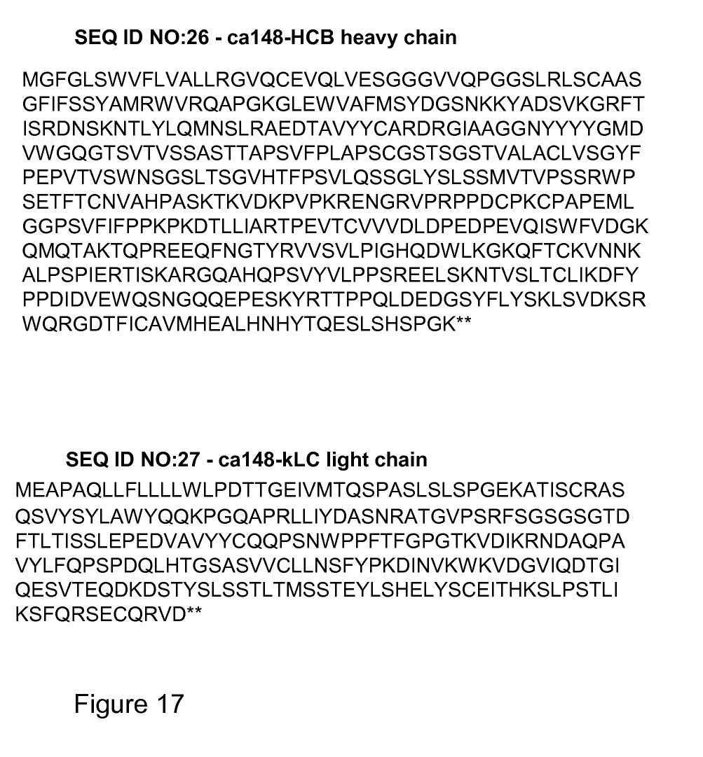

FIG. 16 illustrates a comparison of the caninised and chimeric D2E7 monoclonal antibodies (MAbs) with a further caninised antibody based on anti-human TNF MAb clone 148. The caninised anti-huTNF MAb 148 (SEQ ID NO:26 and SEQ ID NO:27, FIG. 17) was expressed in CHO cells and purified using Protein A chromatography (Panel A, left lane). The caninised 148 MAb was tested for binding to human TNF (Panel B) and canine TNF (Panel C) in comparison to caninised (Ca) and chimeric (Ch) D2E7 based MAbs from FIGS. 14 and 15 (background negative control binding is shown by the arrows). It can be seen from panels B and C that the caninised MAb 148 binds to human TNF, but not canine TNF. Accordingly, the caninised and chimeric MAbs based on D2E7 and the subject of this invention show unexpectedly strong binding to canine TNF equivalent to that to human TNF whereas caninised MAb 148 shows binding to human TNF alone. Therefore, caninised D2E7 based MAbs are surprisingly useful for the treatment of canine diseases mediated by canine TNF.

Taken together, the canine, equine, feline and new human versions of the PETised rat anti-NGF MAb αD11 and the canine PETised versions of the human anti-TNF MAb demonstrate that the PETising conversion of variable domain frameworks by the method described in this patent is robust, reproducible and applicable to multi-species conversion of multiple MAbs.

### Example 4

### Effect of Anti-Canine NGF Monoclonal Antibodies in Reducing Inflammatory Pain In Vivo

### Antibody Therapy

Anti-canine NGF monoclonal antibodies derived from expression vectors expressing SEQ ID NO:7 and SEQ ID NO:70 (canine HCA type heavy chain) were expressed in CHO cells and purified by a combination of ion exchange chromatography, hydrophobic interaction chromatography and size exclusion chromatography and buffer exchanged into phosphate buffered saline.

### Canine Model of Inflammation

All experiments were carried out with prior approval of the Institutional Ethics Committee (CRL, Ireland). Beagle dogs were injected (=day −1) with kaolin into the footpad of one hind leg in order to generate a self-resolving inflammation beginning approximately 24 hours later and which causes the dogs to become temporarily lame. In this model, once the initial inflammation response to kaolin recedes, the dogs become steadily less lame over the period of approximately 1-2 weeks and then make a full recovery.

Groups of 3 dogs were injected intravenously with either anti-canine NGF monoclonal antibodies at 200 μg/kg body weight or phosphate buffered saline as vehicle control (=day 0). The dogs were assessed for lameness over 7 days by a visual scoring method (score 0, no lameness (full weight bearing); score 1, slight lameness (not full weight bearing but walking well); score 2, moderate lameness (slightly weight bearing and not walking well), score 3, severe lameness (not weight bearing)). Observers were blinded to which dogs received which injection.

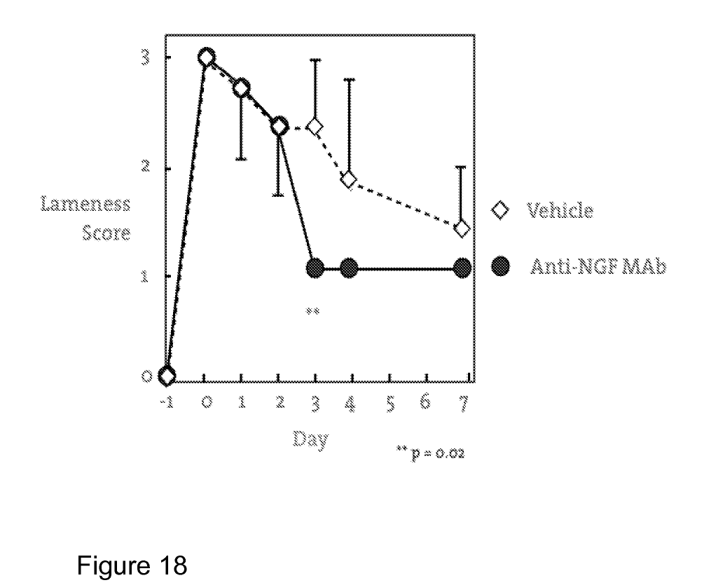

The results are shown in FIG. 18. Lameness scores were reduced in the dogs receiving anti-NGF monoclonal antibodies by day 3 post-injection compared with vehicle control, indicating that the anti-NGF monoclonal antibodies had an effect in reducing the pain in the dogs over that seen with vehicle alone. The delayed activity is consistent with the plasma pharmacokinetics of anti-canine NGF monoclonal antibodies which demonstrated a slow tissue distribution (alpha) phase of approximately 30 hours and the relatively poor vascularisation of the footpad area. The results shown in FIG. 18 show that the anti-canine NGF antibodies produced by the method of the present invention reduce inflammatory pain in dogs with a consequent reduction in lameness.

All documents referred to in this specification are herein incorporated by reference. Various modifications and variations to the described embodiments of the inventions will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1-77. (canceled)

78. A method of modifying a donor immunoglobulin for use in a target species, the method comprising:
determining an amino acid sequence of framework regions of heavy and/or light chain variable domains of a donor immunoglobulin from a species other than the target species, wherein the donor immunoglobulin has binding specificity for a target epitope present in the target species,
comparing each amino acid residue of the amino acid sequence of the framework regions of the heavy and/or light chain variable domains of the donor immunoglobulin with each amino acid residue present at a corresponding position in an amino acid sequence of framework regions of one or more immunoglobulins from the target species, to identify one or more amino acid residues within the amino acid sequence of the framework regions of the heavy and/or light chain variable domains of the donor immunoglobulin that is not present at the corresponding position in the amino acid sequence of the framework regions of at least one of the one or more immunoglobulins derived from the target species, and
substituting the one or more identified amino acid residues present in the amino acid sequence of the framework regions of the heavy and/or light chain variable domains of the donor immunoglobulin, but not present at the corresponding position in the amino acid sequence of the framework regions of at least one of the one or more immunoglobulins derived from the target species, with an amino acid residue which is present at the corresponding position in the amino acid sequence of the framework regions of at least one of the one or more immunoglobulins derived from the target species to obtain a modified donor immunoglobulin,
wherein the modified donor immunoglobulin does not contain any amino acid in any position within the framework regions of the heavy and/or light chain variable domains which would be foreign at that position in one or more immunoglobulins derived from the target species.

79. The method as claimed in claim 78, wherein substitution of an amino acid residue present in the amino acid sequence of the framework regions of the heavy and/or light chain variable domains of the donor immunoglobulin is undertaken using the principle of conservative substitution.

80. The method as claimed in claim 78, further comprising replacing at least one constant domain of the heavy and/or light chain of the donor immunoglobulin with a constant domain of a heavy and/or light chain derived from an immunoglobulin from the target species.

81. The method as claimed in claim 78, wherein the target species is a mammalian target species.

82. The method as claimed in claim 81, wherein the mammalian species is a companion animal selected from the group consisting of a dog, a cat and a horse.

83. The method as claimed in claim 81, wherein the mammalian species is human.

84. A method for treating disease in a subject in need thereof comprising administering to the subject a therapeutically effective amount of the modified donor immunoglobulin produced by the method as claimed in claim 78, or an antigen binding fragment thereof.

85. A neutralizing antibody or an antigen binding fragment thereof which is capable of specifically binding to human nerve growth factor (NGF), wherein the antibody or antigen binding fragment comprises a light chain variable region comprising the amino acid sequence of SEQ ID NO:13 or an amino acid sequence which has an identity of at least 85% thereto and/or a heavy chain variable region comprising the amino acid sequence of SEQ ID NO:14 or an amino acid sequence which has an identity of at least 85% thereto, wherein the antibody or an antigen binding fragment thereof does not contain any amino acid in any position within the framework regions of the heavy and/or light chain variable domains which would be foreign at that position in one or more immunoglobulins derived from a human.

86. The antibody or antigen binding fragment thereof as claimed in claim 85, wherein the light chain comprises the amino acid sequence of SEQ ID NO:25, or an amino acid sequence which has an identity of at least 85% thereto.

87. The antibody or antigen binding fragment thereof as claimed in claim 85, wherein the heavy chain comprises the amino acid sequence of SEQ ID NO:24, or an amino acid sequence which has a sequence identity of at least 85% thereto.

88. A pharmaceutical composition comprising the antibody or antigen binding fragment thereof as claimed in claim 85 and at least one pharmaceutically acceptable diluent or carrier.

89. A method for treating, ameliorating or inhibiting pain in a human in need thereof, the method comprising the step of administering to the human a therapeutically effective amount of the antibody or antigen binding fragment as claimed in claim 85.

90. A method for treating or preventing arthritis in a human, the method comprising the step of administering to the human a therapeutically effective amount of an antibody or antigen binding fragment as claimed in claim 85.


SEQUENCE LISTING (71 sequences)

SEQ ID NO: 1 - protein, 107 aa, Artificial Sequence
  light chain variable domain of a canine version of alpha D11 anti-NGF MAb - canine VK
DIVMTQSPASLSLSQEEKVTITCRASEDIYNALAWYQQKPGQAPKLLIYNTDTLHTGVPSRFSGSGSGTEFSLTISSLEPEDVAVYYCQHYFHYPRTFGAGTKVELK

SEQ ID NO: 2 - protein, 122 aa, Artificial Sequence
  heavy chain variable domain of a canine version of alpha D11 anti-NGF MAb - canine VH
EVQLVESGGDLVNPGGTLTLSCVVSGFSLTNNNVNWVRQALGRGLEWVGGVWAGGATDYNSALKSRLTITRDTSKSTVFLQMHSLRSEDTATYYCARDGGYSSSTLYAMDAWGQGTLVTVSS

SEQ ID NO: 3 - protein, 107 aa, Artificial Sequence
  light chain variable domain of a feline version of alpha D11 anti-NGF MAb - feline VK
DIVMTQTPLSLSVTPGEPASISCRASEDIYNALAWYLQKPGQSPRRLIYNTDTLHTGVPDRFSGSGSGTDFTLRISRVEADDVGVYFCQHYFHYPRTFGPGTKLEIK

SEQ ID NO: 4 - protein, 122 aa, Artificial Sequence
  heavy chain variable domain of a feline version of alpha D11 anti-NGF MAb - feline VH
QVQLVESGGDLVQPGGSLRLTCAASGFSLTNNNVNWVRQAPGKGLEWMGGVWAGGATDYNSALKGRFTISRDNAKNTLYLQMNSLKTEDTATYYCARDGGYSSSTLYAMDAWGQGTLVTVSS

SEQ ID NO: 5 - protein, 107 aa, Artificial Sequence
  light chain variable domain of an equine version of alpha D11 anti-NGF MAb - equine VK
DIVMTQSPASLSASLGETVTIECRASEDIYNALAWYQQKPGQSPKLLIYNTDTLHTGVPSRFSGSGSGTDYSLTINSLQSEDVASYFCQHYFHYPRTFGQGTKLELK

SEQ ID NO: 6 - protein, 122 aa, Artificial Sequence
  heavy chain variable domain of an equine version of alpha D11 anti-NGF MAb - equine VH
QVQLKESGPGLVNPSQTLSLTCTVSGFSLTNNNVNWVRQAPGKGLEWVGGVWAGGATDYNSALKSRATITRDTSKSQVFLQMNSLTSEDTAVYYCARDGGYSSSTLYAMDAWGQGILVTVSS

SEQ ID NO: 7 - protein, 217 aa, Artificial Sequence
  complete light chain of a caninised version of alpha D11 anti-NGF MAb - canine VK and canine kappa light chain constant
DIVMTQSPASLSLSQEEKVTITCRASEDIYNALAWYQQKPGQAPKLLIYNTDTLHTGVPSRFSGSGSGTEFSLTISSLEPEDVAVYYCQHYFHYPRTFGAGTKVELKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 8 - protein, 457 aa, Artificial Sequence
  complete heavy chain of a canine version of alpha D11 anti-NGF MAb - canine VH and canine heavy chain B constant
EVQLVESGGDLVNPGGTLTLSCVVSGFSLTNNNVNWVRQALGRGLEWVGGVWAGGATDYNSALKSRLTITRDTSKSTVFLQMHSLRSEDTATYYCARDGGYSSSTLYAMDAWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFYPPDIDVEWQSNGQQEPESKYRTTPPQLDEDGSYFLYSKLSVDKSRWQRGDTFICAVMHEALHNHYTQESLSHSPGK

SEQ ID NO: 9 - protein, 217 aa, Artificial Sequence
  complete light chain of a feline version of alpha D11 anti-NGF MAb - feline VK and feline kappa light chain constant
DIVMTQTPLSLSVTPGEPASISCRASEDIYNALAWYLQKPGQSPRRLIYNTDTLHTGVPDRFSGSGSGTDFTLRISRVEADDVGVYFCQHYFHYPRTFGPGTKLEIKRSDAQPSVFLFQPSLDELHTGSASIVCILNDFYPKEVNVKWKVDGVVQTKASKESTTEQNSKDSTYSLSSTLTMSRTEYQSHEKFSCEVTHKSLASTLVKSFNRSECQRE

SEQ ID NO: 10 - protein, 457 aa, Artificial Sequence
  complete heavy chain of a feline version of alpha D11 anti-NGF MAb - feline VH and feline heavy chain constant
QVQLVESGGDLVQPGGSLRLTCAASGFSLTNNNVNWVRQAPGKGLEWMGGVWAGGATDYNSALKGRFTISRDNAKNTLYLQMNSLKTEDTATYYCARDGGYSSSTLYAMDAWGQGTLVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 11 - protein, 214 aa, Artificial Sequence
  complete light chain of an equine version of alpha D11 anti-NGF MAb - equine VK and equine kappa light chain constant
DIVMTQSPASLSASLGETVTIECRASEDIYNALAWYQQKPGQSPKLLIYNTDTLHTGVPSRFSGSGSGTDYSLTINSLQSEDVASYFCQHYFHYPRTFGQGTKLELKRDDAKPSAFIFPPSSEELSSGSASVVCLVYGFYPSGATINWKVDGLAKTSSFHSSLTEQDSKDNTYSLSSTLTLPKADYEAHNVYACEVSHKTLSSPLVKSFKRQDC

SEQ ID NO: 12 - protein, 464 aa, Artificial Sequence
  complete heavy chain of an equine version of alpha D11 anti-NGF MAb - equine VH and equine heavy chain 2 constant
QVQLKESGPGLVNPSQTLSLTCTVSGFSLTNNNVNWVRQAPGKGLEWVGGVWAGGATDYNSALKSRATITRDTSKSQVFLQMNSLTSEDTAVYYCARDGGYSSSTLYAMDAWGQGILVTVSSASTTAPKYFQLTPSCGITSDATVALGCLVSDYYPEPVTVSWNSGALTSGVHTFPSVLQSSGLYALSSMVTVPASTWTSETYICNVAHPASSTKVDKRIPPCVLSAEGVIPIPSVPKPQCPPYTHSKFLGGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 13 - protein, 107 aa, Artificial Sequence
  new Hu alpha D11 VL
DIQMTQSPASLSASLGETVTINCRASEDIYNALAWYQQKPGKSPQLLIYNTDTLHTGVPSRFSGSGSGTEYSLKINSLQSEDVASYFCQHYFHYPRTFGGGTKLEIK

SEQ ID NO: 14 - protein, 122 aa, Artificial Sequence
  new Hu alpha D11 VH
QVQLKESGPGLVQPSQTLSLTCTVSGFSLTNNNVNWVRQASGRGLEWMGGVWAGGATDYNSALKSRLTITRDTSKSQVFLKMHSLQSEDTATYYCARDGGYSSSTLYAMDAWGQGTTVTVSS

SEQ ID NO: 15 - protein, 107 aa, Artificial Sequence
  light chain variable domain of a canine version of human D2E7 anti-TNF MAb - canine VK
DIVMTQSPASLSLSQEEKVTITCRASQGIRNYLAWYQQKPGQAPKLLIYAASTLQSGVPSRFSGSGSGTDFTLTISSLEPEDVAVYYCQRYNRAPYTFGQGTKVEIK

SEQ ID NO: 16 - protein, 121 aa, Artificial Sequence
  heavy chain variable domain of a canine version of human D2E7 anti-TNF MAb - canine VH
EVQLVESGGGLVQPGGSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSS

SEQ ID NO: 17 - protein, 217 aa, Artificial Sequence
  complete light chain of a canine version of human D2E7 anti-TNF MAb - canine VK and canine kappa light chain constant
DIVMTQSPASLSLSQEEKVTITCRASQGIRNYLAWYQQKPGQAPKLLIYAASTLQSGVPSRFSGSGSGTDFTLTISSLEPEDVAVYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 18 - protein, 452 aa, Artificial Sequence
  complete heavy chain of a canine version of human D2E7 anti-TNF MAb - canine VH and canine heavy chain A constant
EVQLVESGGGLVQPGGSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 19 - protein, 456 aa, Artificial Sequence
  complete heavy chain of a canine version of human D2E7 anti-TNF MAb - canine VH and canine heavy chain B constant
EVQLVESGGGLVQPGGSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFYPPDIDVEWQSNGQQEPESKYRTTPPQLDEDGSYFLYSKLSVDKSRWQRGDTFICAVMHEALHNHYTQESLSHSPGK

SEQ ID NO: 20 - protein, 454 aa, Artificial Sequence
  complete heavy chain of a canine version of human D2E7 anti-TNF MAb - canine VH and canine heavy chain C constant
EVQLVESGGGLVQPGGSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 21 - protein, 452 aa, Artificial Sequence
  complete heavy chain of a canine version of human D2E7 anti-TNF MAb - canine VH and canine heavy chain D constant
EVQLVESGGGLVQPGGSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFYPPEIDVEWQSNGQPEPESKYHTTAPQLDEDGSYFLYSKLSVDKSRWQQGDTFTCAVMHEALQNHYTDLSLSHSPGK

SEQ ID NO: 22 - protein, 217 aa, Artificial Sequence
  chimeric anti-TNF MAb light chain - human VK and canine kappa light chain
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 23 - protein, 456 aa, Artificial Sequence
  chimeric anti-TNF MAb heavy chain - human VH and canine heavy chain type B constant domain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFYPPDIDVEWQSNGQQEPESKYRTTPPQLDEDGSYFLYSKLSVDKSRWQRGDTFICAVMHEALHNHYTQESLSHSPGK

SEQ ID NO: 24 - protein, 468 aa, Artificial Sequence
  new Hu alpha D11 complete HC-IgG4
MAVLVLLLCLVTFPTCVLSQVQLKESGPGLVQPSQTLSLTCTVSGFSLTNNNVNWVRQASGRGLEWMGGVWAGGATDYNSALKSRLTITRDTSKSQVFLKMHSLQSEDTATYYCARDGGYSSSTLYAMDAWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 234 aa, Artificial Sequence
  new Hu alpha D11 complete kappa LC
MGVPTQLLGLLLLWITDAICDIQMTQSPASLSASLGETVTINCRASEDIYNALAWYQQKPGKSPQLLIYNTDTLHTGVPSRFSGSGSGTEYSLKINSLQSEDVASYFCQHYFHYPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 480 aa, Artificial Sequence
  ca148-HCB heavy chain
MGFGLSWVFLVALLRGVQCEVQLVESGGGVVQPGGSLRLSCAASGFIFSSYAMRWVRQAPGKGLEWVAFMSYDGSNKKYADSVKGRFTISRDNSKNTLYLQMNSLRAEDTAVYYCARDRGIAAGGNYYYYGMDVWGQGTSVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFYPPDIDVEWQSNGQQEPESKYRTTPPQLDEDGSYFLYSKLSVDKSRWQRGDTFICAVMHEALHNHYTQESLSHSPGK

SEQ ID NO: 27 - protein, 238 aa, Artificial Sequence
  ca148-kLC light chain
MEAPAQLLFLLLLWLPDTTGEIVMTQSPASLSLSPGEKATISCRASQSVYSYLAWYQQKPGQAPRLLIYDASNRATGVPSRFSGSGSGTDFTLTISSLEPEDVAVYYCQQPSNWPPFTFGPGTKVDIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 28 - protein, 23 aa, Artificial Sequence
  rat alpha D11 FR1 region of VK light chain
DIQMTQSPASLSASLGETVTIEC

SEQ ID NO: 29 - protein, 23 aa, Artificial Sequence
  caninised FR1 region of VK light chain of rat alpha D11 antibody
DIVMTQSPASLSLSQEEKVTITC

SEQ ID NO: 30 - protein, 23 aa, Artificial Sequence
  felinised FR1 region of VK light chain of rat alpha D11 antibody
DIVMTQTPLSLSVTPGEPASISC

SEQ ID NO: 31 - protein, 23 aa, Artificial Sequence
  equinised FR1 region of VK light chain of rat alpha D11 antibody
DIVMTQSPASLSASLGETVTIEC

SEQ ID NO: 32 - protein, 15 aa, Artificial Sequence
  rat alpha D11 FR2 region of VK light chain
WYQQKPGKSPQLLIY

SEQ ID NO: 33 - protein, 15 aa, Artificial Sequence
  caninised FR2 region of VK light chain of rat alpha D11 antibody
WYQQKPGQAPKLLIY

SEQ ID NO: 34 - protein, 15 aa, Artificial Sequence
  felinised FR2 region of VK light chain of rat alpha D11 antibody
WYLQKPGQSPRRLIY

SEQ ID NO: 35 - protein, 15 aa, Artificial Sequence
  equinised FR2 region of VK light chain of rat alpha D11 antibody
WYQQKPGQSPKLLIY

SEQ ID NO: 36 - protein, 32 aa, Artificial Sequence
  rat alpha D11 FR3 region of VK light chain
GVPSRFSGSGSGTQYSLKINSLQSEDVASYFC

SEQ ID NO: 37 - protein, 32 aa, Artificial Sequence
  caninised FR3 region of VK light chain of rat alpha D11 antibody
GVPSRFSGSGSGTEFSLTISSLEPEDVAVYYC

SEQ ID NO: 38 - protein, 32 aa, Artificial Sequence
  felinised FR3 region of VK light chain of rat alpha D11 antibody
GVPDRFSGSGSGTDFTLRISRVEADDVGVYFC

SEQ ID NO: 39 - protein, 32 aa, Artificial Sequence
  equinised FR3 region of VK light chain of rat alpha D11 antibody
GVPSRFSGSGSGTDYSLTINSLQSEDVASYFC

SEQ ID NO: 40 - protein, 10 aa, Artificial Sequence
  rat alpha D11 FR4 region of VK light chain
FGGGTKLELK

SEQ ID NO: 41 - protein, 10 aa, Artificial Sequence
  caninised FR4 region of VK light chain of rat alpha D11 antibody
FGAGTKVELK

SEQ ID NO: 42 - protein, 10 aa, Artificial Sequence
  felinised FR4 region of VK light chain of rat alpha D11 antibody
FGPGTKLEIK

SEQ ID NO: 43 - protein, 10 aa, Artificial Sequence
  equinised FR4 region of VK light chain of rat alpha D11 antibody
FGQGTKLELK

SEQ ID NO: 44 - protein, 27 aa, Artificial Sequence
  rat alpha D11 FR1 region of heavy chain
QVQLKESGPGLVQPSQTLSLTCTVSGF

SEQ ID NO: 45 - protein, 27 aa, Artificial Sequence
  caninised FR1 region of heavy chain of rat alpha D11 antibody
EVQLVESGGDLVNPGGTLTLSCVVSGF

SEQ ID NO: 46 - protein, 27 aa, Artificial Sequence
  felinised FR1 region of heavy chain of rat alpha D11 antibody
QVQLVESGGDLVQPGGSLRLTCAASGF

SEQ ID NO: 47 - protein, 27 aa, Artificial Sequence
  equinised FR1 region of heavy chain of rat alpha D11 antibody
QVQLKESGPGLVNPSQTLSLTCTVSGF

SEQ ID NO: 48 - protein, 14 aa, Artificial Sequence
  rat alpha D11 FR2 region of heavy chain
WVRQATGRGLEWMG

SEQ ID NO: 49 - protein, 14 aa, Artificial Sequence
  caninised FR2 region of heavy chain of rat alpha D11 antibody
WVRQALGRGLEWVG

SEQ ID NO: 50 - protein, 14 aa, Artificial Sequence
  felinised FR2 region of heavy chain of rat alpha D11 antibody
WVRQAPGKGLEWMG

SEQ ID NO: 51 - protein, 14 aa, Artificial Sequence
  equinised FR2 region of heavy chain of rat alpha D11 antibody
WVRQAPGKGLEWVG

SEQ ID NO: 52 - protein, 32 aa, Artificial Sequence
  rat alpha D11 FR3 region of heavy chain
RLTITRDTSKSQVFLKMHSLQSEDTATYYCAR

SEQ ID NO: 53 - protein, 32 aa, Artificial Sequence
  caninised FR3 region of heavy chain of rat alpha D11 antibody
RLTITRDTSKSTVFLQMHSLRSEDTATYYCAR

SEQ ID NO: 54 - protein, 32 aa, Artificial Sequence
  felinised FR3 region of heavy chain of rat alpha D11 antibody
RFTISRDNAKNTLYLQMNSLKTEDTATYYCAR

SEQ ID NO: 55 - protein, 32 aa, Artificial Sequence
  equinised FR3 region of heavy chain of rat alpha D11 antibody
RATITRDTSKSQVFLQMNSLTSEDTAVYYCAR

SEQ ID NO: 56 - protein, 11 aa, Artificial Sequence
  rat alpha D11 FR4 region of heavy chain
WGQGTTVTVSS

SEQ ID NO: 57 - protein, 11 aa, Artificial Sequence
  caninised FR4 region of heavy chain of rat alpha D11 antibody
WGQGTLVTVSS

SEQ ID NO: 58 - protein, 11 aa, Artificial Sequence
  felinised FR4 region of heavy chain of rat alpha D11 antibody
WGQGTLVTVSS

SEQ ID NO: 59 - protein, 11 aa, Artificial Sequence
  equinised FR4 region of heavy chain of rat alpha D11 antibody
WGQGILVTVSS

SEQ ID NO: 60 - protein, 23 aa, Artificial Sequence
  FR1 region of light chain of a novel humanised variant of alpha D11 (new Hu)
DIQMTQSPASLSASLGETVTINC

SEQ ID NO: 61 - protein, 15 aa, Artificial Sequence
  FR2 region of light chain of a novel humanised variant of alpha D11 (new Hu)
WYQQKPGKSPQLLIY

SEQ ID NO: 62 - protein, 32 aa, Artificial Sequence
  FR3 region of light chain of a novel humanised variant of alpha D11 (new Hu)
GVPSRFSGSGSGTEYSLKINSLQSEDVASYFC

SEQ ID NO: 63 - protein, 10 aa, Artificial Sequence
  FR4 region of light chain of a novel humanised variant of alpha D11 (new Hu)
FGGGTKLEIK

SEQ ID NO: 64 - protein, 22 aa, Artificial Sequence
  FR1 region of heavy chain of a novel humanised variant of alpha D11 (new Hu)
QVQLKESGPGLVQPSQTLSLTC

SEQ ID NO: 65 - protein, 14 aa, Artificial Sequence
  FR2 region of heavy chain of a novel humanised variant of alpha D11 (new Hu)
WVRQASGRGLEWMG

SEQ ID NO: 66 - protein, 32 aa, Artificial Sequence; identical to SEQ ID NO: 52
  FR3 region of heavy chain of a novel humanised variant of alpha D11 (new Hu)

SEQ ID NO: 67 - protein, 11 aa, Artificial Sequence
  FR4 region of heavy chain of a novel humanised variant of alpha D11 (new Hu)
WGQGTTVTVSS

SEQ ID NO: 68 - protein, 453 aa, Artificial Sequence
  complete heavy chain of a caninised version of alpha D11 anti-NGF MAb - VH and HCA
EVQLVESGGDLVNPGGTLTLSCVVSGFSLTNNNVNWVRQALGRGLEWVGGVWAGGATDYNSALKSRLTITRDTSKSTVFLKMHSLQSEDTATYYCARDGGYSSSTLYAMDAWGQGTLVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 69 - protein, 122 aa, Artificial Sequence
  heavy chain variable domain of an alternative canine version of alpha D11 anti-NGF MAb - canine VH
EVQLVESGGGLVQPGGSLRLSCVASGFSLTNNNVNWVRQAPGKGLEWVGGVWAGGATDYNSALKSRFTISRDNAKNTVFLQMHSLRSEDTAVYYCARDGGYSSSTLYAMDAWGQGTSVTVSS

SEQ ID NO: 70 - protein, 453 aa, Artificial Sequence
  complete heavy chain of an alternative caninised version of alpha D11 anti-NGF MAb - VH and HCA
EVQLVESGGGLVQPGGSLRLSCVASGFSLTNNNVNWVRQAPGKGLEWVGGVWAGGATDYNSALKSRFTISRDNAKNTVFLQMHSLRSEDTAVYYCARDGGYSSSTLYAMDAWGQGTSVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 71 - protein, 107 aa, Artificial Sequence
  updated light chain variable domain of a canine version of human D2E7 anti-TNF MAb - canine VK
DIVMTQSPSSLSASQGEKVTITCRASQGIRNYLAWYQQKPGHAPKLLIYAASTLQSGVPSRFSGSGSGTDFTLTISSLEPEDVAVYYCQRYNRAPYTFGQGTKVEIK